(19) United States
(12) Patent Application Publication
(10) Pub. No.: US 2023/0069012 A1
(43) Pub. Date: Mar. 2, 2023

(54) TISSUE REGENERATION PATCH

(71) Applicants: KING'S COLLEGE LONDON, London (GB); UNIVERSITY OF KEELE, Staffordshire (GB)

(72) Inventors: Shukry James HABIB, London (GB); Alicia Jennifer EL HAJ, Stafordshire (GB)

(21) Appl. No.: 17/797,135

(22) PCT Filed: Feb. 8, 2021
(86) PCT No.: PCT/EP2021/052989

§ 371 Date: Aug. 3, 2022

(30) Foreign Application Priority Data
Feb. 7, 2020 (GB) 2001692.9
May 4, 2020 (GB) 2006583.5

(51) Int. Cl. A61L 27/54; A61L 27/24; A61L 27/38; A61L 27/58

(52) U.S. Cl. CPC A61L 27/54; A61L 27/24; A61L 27/3834; A61L 27/58; A61L 2300/252; A61L 2300/412; A61L 2430/34

(57) ABSTRACT

Disclosed is a patch or bandage for tissue regeneration and/or repair. The bandage comprises i) one or more proteins from the Wnt family, or an agonist of the Wnt signalling pathway; and ii) a scaffold, wherein the one or more Wnt proteins or Wnt agonist is immobilised on the scaffold, and wherein the scaffold is formed from a functionalised biocompatible polymer.

### FIELD OF THE INVENTION

The present invention relates to the field of biological tissue repair and regeneration. In particular, the invention relates to the repair and regeneration of tissue, including critical bone fractures, using Wnt immobilised on a scaffold.

### BACKGROUND TO THE INVENTION

With an aging population worldwide, a corresponding increase in the incidence of bone fractures and skeletal diseases represents a substantial burden on healthcare systems (Burge, R. T. et al (2008) J. Medical Economics 4: 51-62; Steiner, C. et al (2002) Eff Clin. Pract. 5: 143-151). In addition, reconstructive surgery following cancer tumour resection and injury, such as in craniofacial trauma, can often require significant donor bone tissue (Hold, P. M. & Prowse, S. J. B. (2011) J. Plast. Reconstr. Aesthet. Surg. 64: 834-835). Approaches such as autologous bone grafts are limited by concerns for donor site morbidity (Shenaq, S. M. (1988) Microsurgery 9: 154-158). Allogenic bone substitutes including demineralised bone matrix or inorganic alloplastic materials are subject to mechanical failure and infection (Lee, K. et al (2005) The Spine Journal 5(6 Suppl): 217S-223S). On the other hand, cell-based therapies aim to deliver stem cells/osteoprogenitors that can respond to the local environment and restore normal function to the injury site (Wozniak, P. & El Haj, A. J. (2007) Tissue Engineering using Ceramics and Polymers. Boccacani, A. R. & Gough J. E. (Eds.) Cambridge; Sharma, P. et al (2005) Applications of Cell Immobilization Biotechnology. Nedovic, V. & Willaert, R. (Eds.) Kluwer Academic Publishers). A major weakness in this approach is that it does not maintain the breadth of the cell differentiation cascade or a source of multipotent precursors that can continue to renew and restore the tissue following transplantation.

Skeletal stem cells (SSC), residing in a niche of the bone marrow, have the ability to self-renew and differentiate into various specialised cell types such as bone cells (Bianco, P. & Robey, P. G. (2015) Development 142: 1023-1027). Self-renewal of stem cells is dependent on external signals; a key signal is the Wnt family of proteins (Garcin, C. L. & Habib, S. J. A. (2017) Results Probl. Cell Differ. 61: 323-350). A hallmark of niche signals is their limited distance of action. Indeed, because of their hydrophobicity resulting from lipid modifications, Wnt proteins are often secreted locally and presented to one side of a responsive cell (Mills, K. M. et al (2017) Open Biology 7: 170140). When Wnt proteins bind to two receptors, Frizzled and Lrp5/6, classical canonical Wnt/β-catenin signalling is activated. This pathway is centred around the accumulation or degradation of β-catenin, which is regulated by a destruction complex that includes glycogen synthase kinase-3 (GSK-3) and adenomatous polyposis coli (APC) among other proteins. β-catenin then translocates into the nucleus and interacts with members of the TCF/LEF family of transcription factors to influence the transcription of target genes. Importantly, Wnt ligands have other receptors and can signal via non-canonical pathways (Garcin & Habib supra).

While powerful genetic tools can be used to manipulate Wnt signalling, studies of hydrophobic Wnt proteins have been impeded by the technical challenges of purification and by their localised action. As a result, Wnt proteins have primarily been used as a target for drug development. One way to address these challenges is to immobilise biologically active Wnt proteins covalently to synthetic surfaces, which enables local signals to be delivered to cells thereby mimicking the cellular niche in vitro (Lowndes, M. et al (2017) Nat. Protoc. 12(7): 1498-1512; Loundes, M. et al (2016) Stem Cell Reports 7: 126-130). In addition, the nature of this technique allows specific spatial targeting of Wnt proteins to any given side of cells and tissues. This technology represents a major breakthrough in tools for understanding Wnt signalling. It has been demonstrated that Wnt3a ligands covalently tethered to microbeads (Wnt3a-beads) can induce oriented asymmetric cell division of single murine Embryonic Stem Cells (ESCs) (Habib, S. J. et al (2013) Science 339: 1445-1448). As a result, the Wnt3a-proximal daughter cells express high levels of nuclear β-catenin and pluripotency genes, whereas the distal daughter cells acquire hallmarks of differentiation towards epiblast stem cell fate. Thus, it has been demonstrated that localised Wnts can affect cell fate decisions in single, mammalian stem cells in vitro by controlling asymmetric cell division, a process that is essential for tissue formation and regeneration. Recently, Wnt3a has been covalently bound onto an aldehyde glass-platform (Wnt3a-platform) (Lowndes, M. et al (2016) Stem Cell Reports 7: 126-137). It has been demonstrated that the Wnt3a-platform is a stable source that can activate Wnt/β-catenin in stem cells and promote long-term self-renewal without the addition of exogenous Wnt3a proteins. The Wnt3a-platform has been developed further into initial 3D models that recreate a human bone niche. hSSCs isolated from adult human bone marrow are a clear choice for engineering autologous bone grafts ex vivo because they have the benefit of scalable isolation and expansion protocols within a growing cell therapy industry (Pittenger, M. F. et al. (1999) Science 284: 143-147; Chippendale, T. et al (2016) Comprehensive Biotechnology 2e, Moo-Young, M. (Ed.), Elsevier; Rafiq, Q. A. et al (2013) Biotechnol. Lett. 35: 1233-1245; Heathman, T. R. J. et al. (2015) Biotechnol. Bioeng. 112: 1696-1707; Ankrum, J. A., et al (2014) Nat. Biotechnol. 32: 252-260). The challenge lies in using hSSCs in a 3D niche that maintains a stem cell population, progenitors and mature bone cells: a differentiation cascade within a biological implant.

Recent work has succeeded in culturing hSSCs on the Wnt3a-platform in a 3D culture environment (Lowndes, M., et al (2016) supra). A 3D environment was generated with the addition of a thin layer of type 1 collagen—the most abundant collagen in bone (Nimni, M. W. et al (1987) J. Biomed. Mater. Res. 21: 741-771)—enabling proliferating cells to migrate away from the platform and into the gel. This technology generated an organised 3D Wnt-Induced human Osteogenic Tissue Model (WIOTM). The WIOTM is formed within a week and consists of Stro1+ hSSCs in close proximity to the Wnt3a source and a multilayer of differentiating cells that show downregulated Stro1 and upregulated Osteocalcin, an osteogenic protein, as cells migrate away from the Wnt3a source. Mineralised nodules were also observed at the upper part of the gel of the WIOTM illustrating that the WIOTM can recapitulate a 3D physiological human bone niche in vitro.

Biomaterial-based regenerative therapies offer the possibility of repairing/replacing non-healing tissues. WO 2019/094617 discloses a bone regeneration product comprising a scaffold, such as polycaprolactone, and a mesenchymal stem cell formulation comprising dense bone regenerating stem cells (DBR-SCs), spongy bone regenerating stem cells (SBR-SCs), or a mixture thereof in particular, in which the product may further comprise a growth factor such as Wnt “ligands”. The scaffold is implanted in or across a bone defect. As a result, the scaffold needs to have a mechanical strength sufficient to handle the load bearing conditions of its implantation, for example during motion (e.g. walking) and/or weight of the body into which the scaffold has been implanted.

WO 2009/131752 addresses poor biological and physiological tolerance of implants and discloses a biological construct comprising a nano-textured biocompatible polymer, such as polycaprolactone, built up in layers. In particular, the implant includes a nanophase surface texture that is capable of temporal, qualitative and qualitative elution of therapeutic agents that mimics the natural healing process of specific tissues. The function of the construct is for the control and differential substance/drug delivery of a therapeutic agent into luminal and abluminal surfaces of tissue to regenerate functional tissue and restore anatomic and physiologic integrity into an organ. The healing process may be augmented by the addition of a tissue-specific, biologically engineered cell sheet which may be overlaid onto the device along with its extracellular matrix. Stem cells are described as being suitable. Each polymeric layer corresponds to a different stage of wound healing and tissue remodelling. Wnt inhibitors may be included to assist with the inflammatory response associated with injury.

WO 2016/112111 discloses a method for regenerating cartilage or bone in which an effective dose of a skeletal stem cell is administered in a matrix. In particular, stem cells are administered together with factors that stimulate differentiation of the stem cells for the regeneration of damaged cartilage or bone. For example, the stem cells may be administered with a Wnt agonist, such as Wnt3a, to induce an osteogenic fate in the cells. The matrix (or lattice) may be a biodegradable polymer such as polycaprolactone. Again, it is the matrix per se that may be transplanted directly into an injury site and so needs to have a degree of structural integrity within the body until the newly formed tissue is able to take over the mechanical load. Injectable pastes and formable putties are disclosed.

WO 2017/0214613 discloses a cell-free medical device that promotes regeneration of skeletal tissue including cartilage and bone. The device has a multi-layered scaffold and a bioactive agent which promotes the homing of stem cells into the device where the stem cells then differentiate, and de novo tissue formation is promoted. Biological factors are adsorbed or coated onto a scaffold material via an unsaturated amine and anionic oligosaccharide.

Chen T. et al (Scientific Reports (2018) 8: 119) evaluated how ageing specifically affects the osteogenic capacity of autologous bone grafts. The study shows that Wnt signalling plays a pivotal role in in the age-related decline of autologous bone grafting efficacy and that autograft efficiency may be restored by ex vivo treatment using a Wnt protein therapeutic. In particular, bone graft materials (cellular marrow and mineralised bone chip fractions) were obtained from mice and incubated with a liposomal formulation of Wnt3a. The bone grafts were then transplanted to the sub-renal capsule (SRC) for 10 days. Ex vivo Wnt3a treatment resulted in increased bone formation in the grafts compared to control.

While in vivo use of scaffolds and stem cells is known for tissue regeneration, together with the importance of localised Wnt signalling, there is still a need for devices that promote efficient and effective bone regeneration, and it is against this background that the present invention has been devised.

### SUMMARY OF THE INVENTION

The present invention encompasses a biological tissue-regeneration patch in which the patch comprises: i) one or more proteins from the Wnt family, or an agonist of the Wnt signalling cascade; and ii) a scaffold, wherein the one or more Wnt proteins or Wnt agonist is immobilised on the scaffold, and wherein the scaffold is formed from a functionalised biocompatible polymer.

The one or more Wnt proteins may be Wnt3, preferably Wnt3a.

Preferably, the one or more Wnt protein, or Wnt agonist, may be immobilised on the scaffold via covalent conjugation. In one example, immobilisation is via primary amine functional groups.

In one embodiment, the biocompatible polymer may be biodegradable.

In another embodiment, the biocompatible polymer may be formed as a film.

Examples of biocompatible polymers include poly (ε-caprolactone) (PCL), poly (lactic-co-glycolic acid) (PLGA), poly (lactide acid) (PLA), Poloxamer, Povidone, polyethylene glycol (PEG) or poly(vinyl alcohol) (PVA).

In another embodiment, the biocompatible polymer is may be functionalised with primary amine groups. Alternatively, the biocompatible polymer may be functionalised with carboxylic acid groups.

The invention also encompasses a patch that further comprises stem cells cultured on the biocompatible polymer. Ideally, the stem cells cultured as a monolayer.

In one embodiment, the stem cells may be overlaid with extra cellular matrix protein. For example, collagen, such as Type-1 collagen, and laminin may be suitable.

In one embodiment, the stem cells may be human mesenchymal or skeletal stem cells (hSSCs). As such, the patch is an osteogenic patch for the regeneration and/or repair of bone.

The invention also encompasses use of the patch for the promotion of endogenous regeneration and/or repair of biological soft tissue, connective tissue or bone. The tissue or bone may have a defect, hole, niche, tear or fracture. Expressed in another way, the invention encompasses a biological tissue endogenous-regeneration method comprising implanting a patch as described herein across, over or around a hole, defect, niche, tear or fracture in soft tissue, connective tissue or bone.

In a particular example, the hole, defect, niche, tear or fracture may be a bone fracture. In another example, the hole may be a craniofacial defect and/or may be due to bone cancer tumour removal. Another example may be use for the repair of one or more cranial holes following brain surgery or stroke rehabilitation. The method and patch of the invention may also be used to grow new bone, for example for leg lengthening or re-growing bone after a section of bone has been removed.

Specific examples of particular orthopaedic uses of the patch of the present invention include Transforaminal Lumbar Interbody Fusion (TLIF) spinal fusion surgery, facet joint single level fusion surgery, segmental tibial defects and high tibial osteotomy.

Also provided is a method of promoting the endogenous repair of biological soft tissue, connective tissue or bone, wherein the method comprises applying a biological tissue-repair patch as described herein to a defect, hole, niche, tear or fracture in the tissue or bone.

In one embodiment the biological tissue-repair patch comprises, or is, a bandage.

In one embodiment the biological tissue-repair patch is applied to the periosteum. When applying the biological tissue-repair patch to a defect, hole, niche, tear or fracture in the tissue or bone, the periosteum may be damaged or discontinuous. In this situation the biological tissue-repair patch is applied so that at least part of it is in contact with at least part of the periosteum. This has the advantage of facilitating endogenous stem cells to reconstitute a therapeutically effective structure at the site of application.

In one embodiment collagen, suitably Type-1 collagen, is applied to the defect, hole, niche, tear or fracture in the tissue or bone before applying, or at the same time as applying, the biological tissue-repair patch. Suitably the collagen, such as Type-1 collagen, is applied by injection, or applied to the wound site as a polymerised gel. This has the advantage of facilitating endogenous stem cells to reconstitute a therapeutically effective structure at the site of application.

In another embodiment the invention relates to a patch as described above for use in treatment of a defect, hole, niche, tear or fracture in biological soft tissue, connective tissue or bone.

In another embodiment the invention relates to a method of treating a defect, hole, niche, tear or fracture in biological soft tissue, connective tissue or bone in a subject, the method comprising applying a patch as described above to said defect, hole, niche, tear or fracture.

The patch also finds use in screening and/or toxicity studies to identify therapeutically effective agents that promote and/or modulate the endogenous regeneration of biological soft tissue, connective tissue or bone.

Also disclosed is a method of manufacturing the biological tissue-regeneration patch as described herein, the method comprising: i) preparing a film from a biocompatible polymer; ii) functionalising the surface of the polymer; and iii) conjugating one or more proteins from the Wnt family or an agonist of the Wnt signalling pathway to the polymer surface.

Preferably, the surface of the polymer may be functionalised to provide primary amine functional groups. Such functionalisation may be achieved by treatment with oxygen plasma and aminopropyl-triethoxysilane (APTES). Alternatively, functionalisation may be achieved by way of activation of carboxylic acid by EDC/NHS reaction.

In a particular embodiment, the method further comprises culturing stem cells after conjugation of Wnt, or an agonist thereof, onto the functionalised polymer surface.

Ideally, the stem cells are cultured as a monolayer and may be cultured for 5-10 days, preferably 7-8 days.

In another embodiment, the method further comprises overlaying a layer of extracellular matrix proteins, such as collagen, such as collagen Type 1, over the stem cells.

### BRIEF DESCRIPTION OF THE DRAWINGS

FIG. 1: Immobilised Wnt3a proteins align the plane of mitotic division and induce asymmetric distribution of Wnt/β-catenin pathway components of bone marrow derived human skeletal stem cells (hSSCs) in 3D-culture model. FIG. 1A: Representative cross section projection of upwards migratory hSSCs in the 3D Wnt-Induced human Osteogenic Tissue Model (WIOTM). Nuclei are stained with 4′,6-diamidino-2-phenylindole (DAPI, yellow). Z=125 μm. FIG. 1B: Schematic representation of oriented division and the osteogenic differentiation process in the WIOTM. FIG. 1C: Quantification of the percentage of hSSCs undergoing perpendicular cell division in Wnt3a- or inactive Wnt3a-WIOTM. Plotted data is mean±s.d. n=3 repeats, N≥15 cells. Stars indicate statistical significance calculated by two-tailed student t test, as follows: *, p<0.05. Representative confocal Z-planes (FIG. 1D and FIG. 1H), 3D reconstructions (FIGS. 1E and 1I) or cross section projection (FIG. 1F and FIG. 1J) of hSSCs undergoing perpendicular (FIGS. 1D to 1G) or parallel (FIGS. 1H to 1K) cell division to the Wnt3a surface. Cells are stained with antibodies against APC (cyan) and β-catenin (magenta). Nuclei are stained with DAPI (yellow). Scale bars, 20 μm. FIG. 1G and FIG. 1K: Fluorescent intensity analysis of APC, ⊕-catenin and DAPI across the Z-axis (axial resolution), expressed as mean fluorescence intensity (arbitrary units). FIG. 1L: Quantification of the distribution of APC and β-catenin in cells dividing perpendicularly or parallel to the Wnt3a surface, following the categories described on the schematics on the right. Data is mean±s.d. n=3 repeats, N≥15 cells.

FIG. 2: Immobilised Wnt3a proteins induce asymmetric distribution of Cadherin-13 (CDH13) and Osteopontin in dividing cells and the WIOTM. FIG. 2A: Cross-section projection image (z=150 μm); and FIG. 2B: Representative confocal Z-planes from the base (cells in direct contact with Wnt3a-surface), middle (10-50 m from surface) and top (50-100 m from surface) of the WIOTM. Cells are stained with antibodies against CDH13 (cyan) and with DAPI (yellow). Scale bar, 100 μm. FIG. 2C: Quantification of osteogenic and stem cell marker expression at three different z-levels in WIOTM. n=3 repeats, N≥90 cells. FIG. 2D and FIG. 2E: Representative confocal images of a series of z-planes illustrating expression of CDH13 (cyan) and OPN (magenta) in FIG. 2D perpendicular and FIG. 2E parallel dividing cells at metaphase. Scale bars, 20 μm. FIG. 2F: Quantification of the distribution of OPN and CDH13 in perpendicular (Perp) and parallel (Para) dividing cells, calculated as described in FIG. 9A. Single cells plotted, bar is mean±s.d., N≥18 cells. Stars indicate statistical significance calculated by two-tailed student t test, as follows: **, p<0.01. Representative 3D reconstruction (FIG. 2G) and 2D side projection (FIG. 2H) of confocal image of daughter cells post cell division. T: Top (Wnt3a-distal) cell, B: Bottom (Wnt3a-proximal cell). Scale bars 20 μm. Representative 3D reconstruction (FIG. 2I) and 2D side projection (FIG. 2J) of confocal image of daughter cells post parallel cell division. T: Top (Wnt-distal) cell, B: Bottom (Wnt3a-proximal cell). L: Left cell, R: Right cell. FIG. 2K: Quantification of the distribution of OPN and CDH13 in perpendicular (Perp) and parallel (Para) post-division cells, calculated as described in FIG. 9A. Cell pairs plotted, bar is mean±s.d., N≥17 cell pairs. Stars indicate statistical significance calculated by two-tailed student t test, as follows: ****, p<0.0001.

FIG. 3: In vitro functional characterisation of Wnt3a-bandage. FIG. 3A: Representative αWNT3A immunoblot of immobilisation process of 20 ng (left) or 60 ng (right) Wnt3a onto PCL film (Wnt3a-bandage). In each immunoblot, Wnt3a input (20 ng or 60 ng), unbound Wnt3a collected after the incubation and three PBS washes (W1, W2, and W3) of the surface on each film are blotted. 0.1% BSA alone was also loaded on the gel as a control. FIG. 3B: Wnt activity of immobilised Wnt3a onto PCL films measured by 7×TCF-luciferase reporter cell line (LS/L) assay. Stars indicate statistical significance as calculated by Tukey test: ***, p<0.001; ****, p<0.0001. FIG. 3C: Wnt-induced eGFP expression and mCherry expression of 7×TCF-eGFP/SV40-mCherry hSSCs on PCL bandage conjugated with 20 ng or 60 ng of Wnt3a. BSA-bandages and inactivated Wnt-bandages were used as negative control. Scale bar, 100 μm. FIG. 3D: Quantification of the Wnt-induced eGFP expression in FIG. 3C. Each bar indicates percentage of eGFP+ cells among mCherry+ cells under each condition (mean±SD, n=5 repeats, N≥555 cells). Stars indicate statistical significance calculated by one-way ANOVA test, as follows: ****, p<0.0001.

FIG. 4: In vivo functional evaluation of Wnt3a-bandage and WIOTM-bandage in the repair of critical size defects of murine calvarial bone. FIG. 4A: Schematic illustration of the surgical procedure followed. Briefly: 4 mm defects were made in either one or both sides of the parietal bone and a functionalised bandage implant overlaid and secured (or no implant for defect-only controls). FIG. 4B and FIG. 4C: Representative CT scan images of calvarial defects in mice transplanted with no implant (Defect only) and transplanted with inactivated-Wnt3a (iWnt) bandage, inactivated-Wnt3a bandage cultured with hSSCs and overlaid with Collagen type 1 (iWnt-hSSCs), active-Wnt3a (Wnt) bandages and WIOTM bandages (FIG. 4C) at eight weeks post-transplantation. FIG. 4D: Quantification (mean s.d., n≥9 animals) of the new bone coverage over the defect site as imaged by CT scanning. Stars indicate statistical significance calculated by one-way ANOVA test, as follows: ns, not significant; *, p<0.05; ***, p<0.01; ****, p<0.001. Hematoxylin and Eosin (FIG. 4E) and Movat's Pentachrome (FIG. 4F) staining of sections showing the regenerated new bone (NB), fusion of host and new bone, host bone (HB) and connective/stromal tissue (CST). Scale bars, 100 m (FIG. 4E) and 500 μm (FIG. 4F). BV: Blood Vessel; BT: Brain Tissues.

FIG. 5: In vivo characterisation of new bone formation in critical size defect of calvarial bone driven by WIOTM, Wnt3a and iWnt3a-hSSCs bandages at 8 weeks post-implant. FIG. 5A: Quantification (Method in FIG. 16A) of the density of total Sclerostin (SOST) positive cells in areas of new and host bone. Mean±s.d., N≥125 cells from 3 animals. Statistical significance as determined by unpaired t-test between all columns: ns, p>0.05. FIG. 5B: Quantification of the percentage of total SOST+ cells in new bone of human origin in WIOTM-bandage and iWnt3a-bandage+ hSSCs as determined by co-expression of human marker (mCherry/hBMG2) and SOST. Note: using the same samples as (B). Mean±s.d., n=3 animals, N≥38 cells. Statistical significance as determined by unpaired t-test: *, p<0.05. FIG. 5C: Micro-CT (μCT) and Brightfield images of new bone (NB) in the defect site. ROI1 is new bone in the middle, and ROI2 is new bone at the edge of the defect, near host bone (HB). Scale bar 250 μm. Asterisk marks mounting artifact. FIGS. 5D, FIG. 5E and FIG. 5F: Representative immunofluorescence images of defect site sections: (D) implanted with WIOTM-bandage, with cells expressing the 7×TCF-eGFP//SV40-mCherry reporter, (E) Wnt3a-bandage alone and (F) inactive Wnt3a-bandage with hSSCs and overlaid with Collagen type 1 (iWnt3a-bandage+ hSSCs). Sections stained with DAPI (yellow), antibodies against Sclerostin (cyan) and the human markers (D) mCherry (red), (F) hBMG2 (red). Solid white box indicates a human marker+ cell, dashed box indicates human marker-. Asterisk marks non-cellular artifact. Scale bars 20 μm.

FIG. 6: In vivo characterisation of 8 weeks post-implant of connective/stromal-like tissue surrounding new bone formed in critical size defects of calvarial bone treated with WIOTM, Wnt3a and iWnt3a-hSSCs bandages. FIG. 6A: Quantification of the percentage of GFP+ human cells and GFP− human cells of total cells found proximal (Prox, <20 μm), M (Middle): 50% of the remaining thickness of the tissue; D (Distal): the other 50% of the remaining thickness of the tissue. Mean±s.d., n≥3, N≥318 cells from 3 animals, percentages displayed on chart. FIG. 6B: Representative immunofluorescence images of defect site and host tissues implanted with WIOTM-bandage with cells expressing the 7×TCF-eGFP//SV40-mCherry. Sections stained with DAPI (white) and antibodies against mCherry (red) and GFP (green). Arrows: blue: mCh−/GFP−; magenta: mCh+/GFP−; white: mCh−/GFP−. Scale bars 50 μm. HB: Host Bone, Peri: Periosteum. Scale bars 50 μm. FIG. 6C and FIG. 6D: Representative immunofluorescence images of defect site and host tissues: FIG. 6C: Representative immunofluorescence images of defect site implanted with WIOTM-bandage with cells expressing the 7×TCF-eGFP//SV40-mCherry reporter. FIG. 6D: Wnt3a-bandage alone and inactive Wnt3a-bandage with hSSCs overlaid with Collagen type 1 (iWnt3a-bandage+ hSSCs). Sections stained with DAPI (yellow), antibodies against CDH13 (cyan) and in (C) the human marker mCherry (red). Arrows: white: mCh+/CDH13+; blue: mCh−/CDH13+; magenta: mCh+/CDH13−; yellow: mCh−/CDH13−. Scale bars 50 μm. P: Periosteum, S: Suture, HB: Host Bone, Peri: Periosteum, NB: New Bone. FIGS. 6E to 6G: Quantification of cell subpopulations in the defect sites as percentage of total cells in defect sites of (FIG. 6E) WIOTM-bandage (WIOTM-bandage) with cells expressing the 7×TCF-eGFP//SV40-mCherry reporter, (FIG. 6F) Wnt3a-bandage alone and (FIG. 6G) inactive Wnt3a-bandage with hSSCs overlaid with Collagen type 1 (iWnt3a-bandage+ hSSCs), and host tissues. Proximal, <20 μm), Middle: 50% of the remaining thickness of the tissue; Distal: the other 50% of the remaining thickness of the tissue to the bandage surface. Mean s.d., n≥3, N≥25 cells from 3 animals, percentages displayed on chart. P: Periosteum, S: Suture, HB: Host Bone. Peri: Periosteum, NB: New Bone. (FIG. 6H) Statistical significance of comparisons of data shown in (FIGS. 6E to 6G), as determined by unpaired t-test: ns, p>0.05; *, p<0.05; **, p<0.01; *** p<0.001.

FIG. 7: Immobilisation of Wnt on PLGA bandage as demonstrated by Western blot and activation of Wnt/beta-catenin pathway in Wnt-reporter cell line. FIG. 7A: Quantification of percentage area of bone repair of a unilateral 4 mm diameter critical sized calvarial defect after 8 weeks, in 13 week old female SCID (severe combined immunodeficient) mice. Drilled defects were overlaid with collagen. In Defect Only (n=14), no further treatment was given. In the PLGA-WIOTM condition (n=10), poly(lactic-co-glycolic acid) (PLGA) Wnt3a-bandages were seeded with hSSCs and cultured for 7 days in a 3D osteogenic matrix in vitro prior to transplantation, forming the Wnt-induced human osteogenic tissue model (WIOTM). Statistical significance calculated by unpaired two-tailed T-test. FIG. 7B: Representative images of micro-Computed Tomography (microCT) scans of the calvarial defects 8 weeks following surgery. Yellow dashed ellipse designates the original 4 mm diameter defect size. Scale bars are 2 mm. FIG. 7C: Fold change of luciferase activity as measured by Wnt/β-catenin reporter LSL cells harbouring the 7×TCF-luciferase construct, seeded on PLGA bandages or tissue culture plastic. Readings from the conditions: Wnt3a-PLGA (60 ng input Wnt3a immobilised on PLGA), Media Control (tissue culture plastic and standard culture media), and Soluble Wnt3a (tissue culture plastic and media supplemented with 45 ng soluble Wnt3a) were normalised against BSA-PLGA (Bovine Serum Albumin immobilised on PLGA). n=9, pooled from 3 independent experiments. Statistical significance calculated by unpaired two-tailed T-test. FIG. 7D: Representative Western blot showing 60 ng Wnt3a (equivalent to the amount input at the immobilisation step), Unbound Wnt3a (collected following immobilisation), Washes 1-3, and BSA only (Bovine Serum Albumin). Molecular weight ladder in kilo Daltons (kD).

FIG. 8: The axis of mitotic division of hSSCs in 3D culture and the cellular distribution of aPKC ζ and Numb. FIG. 8A and FIG. 8B: Representative images of hSSCs dividing parallel (A) or perpendicular (B) to the Wnt-surface, stained with antibodies against the centrosomal marker centrin1 (magenta), or DAPI (yellow). Inset is magnification of white box. Arrow indicates centrosomes. Scale bars, 50 μm. FIG. 8C: Quantification of the distribution of aPKCζ in cells dividing perpendicularly or parallel to the Wnt3a surface, categorised as in FIG. 1L. N≥15 cells. FIG. 8D and FIG. 8E: Representative confocal Z-planes and 3D reconstruction of hSSCs undergoing perpendicular (FIG. 8D) or parallel (FIG. 8E) cell division. Cells are stained with antibodies against α-Tubulin (cyan), aPKCζ (magenta), and DAPI (yellow). Scale bars 20 μm. FIG. 8F and FIG. 8G: Representative confocal Z-planes of hSSCs undergoing perpendicular (FIG. 8F) or parallel (FIG. 8G) cell division. Cells are stained with DAPI (yellow) and antibodies NUMB (red). Scale bars 20 μm. FIG. 8H and FIG. 8I: 3D reconstructions and 2D projection of the perpendicular and parallel dividing cells. Note that the cell in FIG. 8F and FIG. 8H is shown again in FIGS. 10D to 10F, with co-staining. FIG. 8J: Quantification (Single cells plotted, bar is mean±s.d, N≥10 cells) of the NUMB distribution in perpendicular (Perp) or parallel (Para) dividing cells, as depicted in FIG. 9A.

FIG. 9: The distribution of cell fate markers during cell division and multipotency of cultured hSSCs. FIG. 9A: Schematic representation of the quantification protocol for perpendicular or parallel dividing cells. Briefly, DAPI staining were used as middle reference, and mean fluorescence intensity of marker 1 and marker 2 are measured for bottom and top parts of the cell from DAPI midline (in perpendicular division cells) or for right and left parts of the cells from DAPI midline (in parallel dividing cells). The ratio between marker 1 and 2 is calculated for each cell half, and the fold difference between the ratios is plotted. FIG. 9B and FIG. 9C: Representative confocal Z-planes of perpendicular (FIG. 9B) or parallel (FIG. 9C) dividing cells at telophase, stained with antibodies against CDH13 (cyan) and OPN (magenta), and DAPI (yellow). Scale bar, 20 μm. FIG. 9D: In vitro differentiation of hSSCs (passage 5) at day 14 of culture in Adipogenic, Chondrogenic and Osteogenic media. Upper row: immunofluorescent staining for markers of the three lineages (red) (FABP4, Aggrecan and Osteocalcin (OCN), respectively and DAPI (blue). Lower row: cells stained without primary antibody to show specificity. Scale bars 150 m.

FIG. 10: The distribution of cell fate markers in the WIOTM and during hSSC division. FIG. 10A: Cross-section image projection (z=120 μm) and FIG. 10B: representative confocal Z-planes from the base (cells in direct contact with Wnt3a-surface), middle (10-50 μm from surface) and top (50-100 μm from surface) of the WIOTM. Cells are stained with antibodies against PLXNA2 (cyan) and OPN (magenta) and with DAPI (yellow). Scale bar 100 μm. FIG. 10C: Quantification of osteogenic and stem cell marker expression at three different z-levels in WIOTM. Data is mean±s.d., n=3 repeats, N≥262 cells. FIG. 10D: Representative confocal Z-planes of a hSSC undergoing perpendicular cell division, stained with antibodies against PLXNA2 (cyan), OPN (magenta), and DAPI (yellow). Note this cell is also shown in FIGS. 8F and 8H. Scale bars 20 μm. FIG. 10E: 3D reconstructions of the cell shown in FIG. 10D. FIG. 10F: Enlargement of the 3D reconstructed DAPI signal in the top-down view of FIG. 10E. FIG. 10G: Quantification of the distribution of OPN and PLXNA2 in perpendicular (Perp) and parallel (Para) dividing cells, calculated as described in FIG. 9A. Single cells plotted, bar is mean±s.d., N≥17 cells Stars indicate statistical significance calculated by two-tailed student t test, **, p<0.01.

FIG. 11: The distribution of cell fate markers during hSSC division. 3D reconstructions of representative post-mitotic cells of perpendicular (T: top, B: bottom) (FIG. 11A) and parallel divisions (L: left, R: right) (FIG. 11B). Cells were stained with DAPI (yellow) and PLXNA2 (cyan). FIG. 11C: Quantification of the distribution of OPN and PLXNA2 in perpendicular and parallel divided post-mitotic cells, calculated as described in FIG. 9A. Cell pairs plotted, bar is mean s.d., N≥17 cells Stars indicate statistical significance calculated by two-tailed student t test, as follows: ***, p<0.001.

FIG. 12: The distribution of cell fate markers in the WIOTM and during hSSC division. FIG. 12A: Representative maximum intensity Z-projections of confocal images of the base (z=0-10 m) or top (z=50-60 m) of the WIOTM depicting the change of protein expression in upwards migratory cells. Cells are stained with antibodies against OCN (cyan), STRO1 (magenta), and DAPI (yellow). Scale bar, 50 μm. FIG. 12B and FIG. 12C: Representative confocal Z-planes of hSSCs undergoing perpendicular (FIG. 12B) or parallel (FIG. 12C) cell division. Cells are stained with antibodies against OCN (cyan), Stro1 (magenta), and DAPI (yellow). Scale bars 20 μm. FIG. 12D and FIG. 12E: Quantification of the distribution of OPN and PLXNA2 in perpendicular and parallel orientated dividing (FIG. 12D) and post-mitotic cells (FIG. 12E), calculated as described in FIG. 9A. Single cells (D) or cell pairs (E) plotted, bar is mean s.d., N≥17 & 18 cells/cell pairs respectively. Stars indicate statistical significance calculated by two-tailed student t test, as follows: **, p<0.01. 3D reconstruction (FIG. 12F) and 2D projection (FIG. 12G) of a representative perpendicular post-mitotic cell pair. T: Top cell, B: Bottom cell.

FIG. 13: functionalisation of PCL films for Wnt immobilisation. FIG. 13A: Schematic illustrating the oxidation of the PCL surface using O₂ plasma treatment and the secondary covalent conjugation of (3-aminopropyl)triethoxysilane (APTES) with the radical hydroxyl group to provide stable primary amine. FIG. 13B: Quantification of the fluorescein isothiocyanate FITC conjugation to characterise the efficacy of primary amine functionalisation and the stable physical integrity of PCL over 8 weeks in culture. PCL was treated with hexamethyldiamine (HMDA), O₂ plasma or Air prior to Wnt immobilisation. Results are mean±s.d., expressed in arbitrary units. Stars indicate statistical significance calculated with one-way ANOVA, as follows: ****, p<0.001; ns, not significant. FIG. 13C: Standard curve and linear fit for dissolved FITC absorbance at 500-550 nm. FIG. 13D: Estimation of number of amine sites on PCL by extrapolation from bound FITC absorbance (FIG. 13B) to equivalent absorbance of dissolved FITC (FIG. 13C).

FIG. 14: Immunohistochemistry of connective/stromal-like tissues of host and in the WIOTM-bandage treated calvarial defect site. FIG. 14A: Micro-CT (pCT) scan and immunohistochemical staining of new bone (NB) and host bone (HB) with antibodies against STRO1 (FIG. 14B) and CDH13 (FIG. 14C). Enlargements show an area of host bone and periosteum (1), new bone formed near the drilled defect edge (2) and new bone formed in the centre of the defect (3). Black arrows indicate the original edges of the drilled defect. C is connective/stromal-like tissue, P is periosteum, S is Suture. Nuclei are counterstained with Hematoxylin.

FIG. 15: Immunohistochemistry of calvarial bone. FIG. 15A: Immunohistochemical (IHC) staining for OCN, OPN and PLXNA2 areas of new bone (NB) in defects treated with WIOTM-bandage and host bone (HB) from the same samples. C: connective tissue, P: periosteum. Scale bars 50 μm. FIG. 15B: Immunohistochemical staining Immunohistochemical staining for OCN, OPN and CDH13 for areas of new bone (NB) in defects treated with Wnt3a-bandage and host bone (HB) from the same samples. C: connective tissue, P: periosteum. Scale bars 50 μm. FIG. 15C: Immunohistochemical staining of the sagittal suture (S) with antibodies against OCN, OPN, PLXNA2, STRO1 and CDH13. Black dotted line outlines host suture. HB: host bone. Scale bars 50 μm. FIG. 15D: Negative controls for IHC. Upper row: regions of tissue, stained following standard protocol, but without primary antibody. Lower row: isotype controls for antibodies used in FIG. 15A to 15C and FIGS. 14B and 14C, as follows: Mouse IgG1 (OPN isotype), Rabbit IgG (CDH13, OCN, PLXNA2 isotype) and Mouse IgM (Stro1 isotype). S: Suture, HB: host bone, NB: new bone, C: connective tissue. Scale bars 50 μm.

FIG. 16: Immunofluorescence Quantification Method Schematics, FIG. 16A: For the quantification of bone cells. (I) An area of stained host tissue (i.e. host bone) is selected, and the highest intensity cellular signal for the marker of interest is determined (II). (III) This intensity is used to threshold the sample tissue (e.g. new bone), such that all remaining signal is of a higher intensity than the control tissue (IV). Positive cells can then be quantified. FIG. 16B: For the quantification of cells in connective tissue (I). (II) An area of stained host tissue (i.e. host periosteum) is selected, and the highest intensity cellular signal for the marker of interest is determined. (III) This intensity is used to threshold the sample tissue (e.g. connective tissue in the defect), such that all remaining signal is of a higher intensity than the control tissue (IV). Positive cells can then be quantified. For analysis in areas under a bandage treatment, regions are determined as follows: P (Proximal): <20 μm from the bandage surface; M (Middle): 50% of the remaining thickness of the tissue; D (Distal): the other 50% of the remaining thickness of the tissue. FIG. 16C: Representative immunofluorescence images of tissue treated with WIOTM-bandages containing 7×TCF-eGFP//SV40-mCherry cells and host tissues, stained with DAPI (yellow), and antibodies against the human markers mCherry (red) and hBMG2 (cyan), with significant co-localization of signal. White arrows show mCherry+/hBMG2+ cells. Scale bars 50 μm.

FIG. 17: In vivo characterization of 8 weeks post-implant of connective/stromal-like tissue surrounding new bone formed in critical size defects of calvarial bone treated with WIOTM-bandage, Wnt3a-bandage and iWnt3a-bandage+ hSSCs. FIG. 17A: Quantification (FIG. 16B) of the density of total (DAPI) and CDH13 positive cells in bandage-treated defect sites (N≥44 cells) and host tissues (N≥12 cells). Mean s.d., all n≥3, from 3 animals. Statistical significance as determined by unpaired t-test between all columns: *, p<0.05; ns, p>0.05. FIG. 17B: Quantification of cell subpopulations in the defect sites as percentage of total cells in defect sites of WIOTM-bandage with cells expressing the 7×TCF-eGFP//SV40-mCherry reporter and host tissues. Proximal (Prox, <20 μm), middle (Mid) and distal (Dist) (50% remaining tissue thickness each) from the bandage surface. Mean±s.d., n≥3, N≥16 cells from 3 animals, percentages displayed on chart. FIG. 17C: Representative immunofluorescence images of defect site under WIOTM-bandage treatment containing 7×TCF-eGFP//SV40-mCherry cells, and host tissues, stained with DAPI (yellow), and antibodies against PLXNA2 (cyan) and the human marker mCherry (red). Arrows: white: mCh+/PLXNA2+; blue: mCh−/PLXNA2+; magenta: mCh+/PLXNA2−; yellow: human−/Stro1−. Prox: proximal, Mid: middle, Dist: distal, P: Periosteum, S: Suture, HB: Host Bone. Scale bars 50 μm. FIG. 17D: Quantification of the density of total (DAPI) and Stro1 positive cells in bandage-treated defect sites (N≥36 cells). Mean±s.d., all n=3 animals. Statistical significance as determined by unpaired t-test between all columns: *, p<0.05; ns, p>0.05. FIG. 17E: Representative immunofluorescence images of defect site sections implanted with WIOTM-bandage with cells expressing the 7×TCF-eGFP//SV40-mCherry reporter and inactive Wnt3a-bandage with hSSCs. Sections stained with DAPI (yellow), antibodies against Stro1 (cyan) and the human markers mCherry or hBMG2 (red). Arrows: white: human+/Stro1+; blue: human−/Stro1+; magenta: human+/Stro1−; yellow: human−/Stro1−. Asterisk shows non-cellular unspecific staining. Scale bars 50 μm.

### DETAILED DESCRIPTION OF THE INVENTION

The role of Wnt/β-catenin signalling pathway in orchestrating bone regeneration is well documented in various models (Doro, D. H. et al (2017) Front Physiol. 8: 956; Zhao, H. et al (2015) Nat. CellBiol. 17: 386-396; Maruyama, T. et al (2016) Nat. Commun. 7: 10526; Wilk, K. et al (2017) Stem Cell Reports 8: 933-946; Zhou, H. & Lee, J. (2011) Acta Biomater. 7: 2769-2781; Tan, S. H. et al (2014) Proc. Nat. Acad Sci. U.S.A. 111: E5262-71; Krause, U. et al (2010) Proc. Nat. Acad Sci. U.S.A. 107: 4147-4152; Baron, R. & Kneissel, M. (2013) Nat. Med 19: 179-195; Li, S. et al (2015) PLoS ONE 10: e0138059; Minear, S. et al (2012) Sci. Transl. Med 2: 29ra30). Aberration in Axin2 expression, which serves as the critical negative regulator for Wnt/β-catenin signalling pathway, was found to accelerate proliferation and earlier differentiation of progenitor cells in the mouse model (Tan, S. H. et al (2014) supra; Li, S. et al (2015) PLoS ONE 10: e0138059). The lineage tracing in the mouse model further delineates the Wnt-induced osteogenic regeneration upon injury, where the Wnt-responsive stem cell population resides in the suture mesenchyme is found to be the key SSCs in driving expansion and differentiation (Zhao, H. et al (2015) supra; Maruyama, T. et al (2016) supra).

Pharmacological studies have been developed to explore the therapeutic efficacy of Wnt proteins in promoting bone regeneration. Wnt3a delivered by liposomal vesicles was identified to stimulate proliferation of skeletal progenitor cells, and thus accelerated osteoblast differentiation that is key for bone growth (Minear, S. et al (2010) Sci. Transl. Med 2(29): 29ra30). However, this protein-based approach relies on the non-targeted diffusion of the liposome in the injury site to reach Wnt-responsive cells that will promote/enhance bone growth. Therefore, even though promising bone growth was identified, the regeneration rate was lower than the conventional osteoconductive implant that serves as a gap-filling tool. Other small molecule approaches aim to modulate Wnt/β-catenin signalling pathway in favour of osteogenesis by inhibiting glycogen-synthetase-kinase-30 (GSK30), and peroxisome proliferator-activated receptor-7 (PPARγ) was found to be effective in osteoinductive therapy (Krause, U. et al(2010) Proc. Natl. Acad. Sci. U.S.A. 107: 4147-4152; Zeitouni, S. et al(2012) Sci. Transl. Med. 4(132): 132ra55). Although promising candidates, small molecules are often nonspecific and can activate/inhibit other signalling pathways (Lowndes, M. et al (2017) supra). Additionally, GSK30 is involved in numerous cellular processes and signalling pathways that are not under the control of Wnt-ligands (Beurel, E. et al (2015) Pharmacol. Ther. 148: 114-131).

Even though pharmacological approaches using soluble drug delivery demonstrated the significant therapeutic effect, it also poses biosafety concern. The soluble drug delivery approach might require multiple rounds of injections in the injured site to achieve the desired effect, which will increase the risk for infection. The lack of specificity and localised action of these agents might trigger collateral damage such as activating/suppressing the response of undesired cell population(s). As a result, the architecture and function of the target tissue, and/or adjacent tissues that can receive the drug by diffusion, might be negatively affected. For example, over activation of Wnt signalling can result in increased bone volume, abnormal bone density and pathological thickening of the bone (Morvan, F. et al (2006) J. Bone Miner. Res. 21: 934-945; Little, R. D. et al (2002) Am. J Hum. Genet. 70: 11-19. Boyden, L. M. et al (2002) N. Engl. J. Med. 346: 1513-1521; Babij, P. et al (2003) Miner. Res. 18: 960-974).

Tumorigenesis is also a concern in patients that are prone to cancer. In many cancers, tumorigenic cells have mutations in downstream effectors of the Wnt/β-catenin pathway that activate the signalling cascade (Zhan, T. et al (2017) Oncogene 36: 1461-1473). Administered agents that can reach these cells, by diffusion, and further activate the Wnt/β-catenin pathway are inherently categorised as high risk in tumorigenesis. Therefore, a targeted delivery of the therapeutic agent that can lead to a controlled and localised activation of Wnt/β-catenin pathway to the target defect site is essential for limiting the side effects and promoting healing. The biological tissue regeneration patch or bandage of the present invention meets these criteria.

The patch/bandage delivers its therapeutic effect by anchoring Wnt proteins, an agonist of the Wnt signalling pathway, or an R-spondin protein, onto a scaffold. R-spondin (Rspo) proteins work synergistically with Wnt proteins to enhance signaling levels but do not activate the signalling pathway in the absence of Wnt proteins themselves. In particular, Rspo proteins are believed to increase receptor availability by inhibiting receptor recycling. The engineering design of the invention mitigates the probability of spontaneous protein leakage and the subsequent side effect of activating other signalling pathways or undesired cell population, as well the risk of carcinogenesis, thus increasing the potential of the translational application.

The Wnt signalling pathway is activated by the binding of a Wnt-protein ligand to a Frizzled family receptor, which passes the biological signal to the Dishevelled protein inside the cell. The canonical Wnt pathway leads to regulation of gene transcription and is thought to be negatively regulated in part by the SPATS1 gene (Janda, C. Y. et al(2012). Science 337(6090): 59-64). The noncanonical planar cell polarity pathway regulates the cytoskeleton that is responsible for the shape of the cell and the noncanonical Wnt/calcium pathway regulates calcium inside the cell.

Anchoring of the Wnt proteins (or agonists or Rspo proteins) is ideally via covalent conjugation with Wnt (or an agonist thereof or Rspo protein) binding covalently to primary amine functional groups on the surface of a biocompatible polymer. Expressed in another way, the surface of the biocompatible polymer is functionalised to present primary amine groups which are used to bind Wnt (or an agonist thereof or Rspo protein) by covalent binding.

In some situations, particularly in connective tissue, the use of antagonists of the Wnt signalling pathway, such as DKK1, may be desirable to inhibit activation of the pathway in specific cells such as fibroblasts.

The term “polymer” refers to when a molecule formed from the union of multiple (two or more) monomers. The polymer may be preferably amphipathic, and may be organic, semi-synthetic, or synthetic. Examples of polymers relevant to the present invention include, but are not limited to biologically tolerated and pharmaceutically acceptable polymers that are approved by agencies such as the US Federal Drug Agency and include polycaprolactone (“PCL”), poly(lactic-co-glycolic acid) (“PLGA”), poly(l-lactic acid) (“PLA”), poly(glycolic acid) (“PGA”), Poloxamer, Povidone, polyethylene glycol (PEG), Polydioxanone (PDS), Poly(vinyl alcohol) (PVA), poly(ether urethane), Dacron, polytetrafluorurethane, polyurethane (“PU”), poly(glycolide-co-caprolactone) (PGCL), Poly(l-lactide-co-ε-caprolactone) PLCL, poly-L/D-lactic acid (PLDLA), alginate and/or silicon.

The polymer may also include naturally occurring materials such as collagen I, collagen III, fibronectin, fibrin, laminin, cellulose ester, or elastin.

Ideally the polymer is biodegradable as this allows the patch to be implanted without the need for additional surgery to remove the patch. The patch may be biodegradable over a period of time that is sufficient simply to promote endogenous repair, or the patch may biodegrade over a longer period of time to provide mechanical support to newly formed tissue. For example, the in vivo degradation rate of PCL is slow and might require a couple of years (Sun, H. et al (2006) Biomaterials 27: 1735-1740) while the in vivo degradation rate of the Poly Lactic-co-Glycolic Acid (PLGA) can be as short as one month (Makadia, H. K. & Siegel, S. J. (2011) Polymers (Basel) 3: 1377-1397). Since the patch is overlaid onto a defect site, it can be easily removed after the repair is completed to avoid potential side effects of the polymer.

It will be appreciated that the polymer film may be formed by any appropriate method. Where the polymer is PCL, for example, methods for making films are well known. For example, the film may be formed by moulding, 3D printing or electrospinning.

In one embodiment, the polymer film may be embedded within another film such as a hydrogel film, so as to allow patterning of a larger (hydrogel) film with different ligands, for example, Wnt3a nanoparticles on one side and DKK1 nanoparticles on the other side to increase differentiation.

To enable Wnt (or an agonist thereof) to be immobilised on the polymer surface, the polymer surface may be functionalised to add suitable binding groups. In particular, the surface may be functionalised with primary amines to provide a covalent linkage with Wnt. Alternative linkages include but are not limited to carboxylic acid that can be converted to succinimide ester, and glutaraldehyde. Covalent linkage is preferred because maintains Wnt at the specific location, which has been shown to enhance the regenerative effect of Wnt.

Described above is an acellular patch or bandage. In a particular embodiment, the patch or bandage may further comprise stem cells cultured on the biocompatible polymer, thereby providing a 3-dimensional (3D) cellular bandage or patch. Ideally the cells are cultured on the functionalised polymer as a monolayer.

The inventors have found that overlaying the stem cells with a layer of extracellular protein, such as collagen, promotes the migration of the stem cells away from the Wnt source and towards the site of injury for repair. If the patch is to be used, for example, for the regeneration of bone, type I collagen is particularly appropriate as this variant is most abundant in bone. After one week in osteogenic media, a Wnt-induced osteogenic tissue forms where hSSCs are maintained close to the Wnt-source and a cascade of increasingly differentiating osteogenic cells migrate into the 3D hydrogel and away from the Wnt source.

In one example, the stem cells may be human skeletal stem cells (hSSCs) and the bandage is an osteogenic patch. It will be appreciated that the primary human skeletal stem cells (hSSCs) may be isolated from any suitable source, including bone marrow, adipose tissue or made from induced pluripotent stem cells (iPSCs).

It will be appreciated that the cellular bandage or patch may be used for the promotion of endogenous regeneration of biological soft tissue, connective tissue or bone, such as for regeneration and/or repair of a niche, tear or fracture in biological soft tissue, connective tissue or bone. It will be appreciated that the invention is applicable to any Wnt-responsive stem cells, including mesenchymal or epithelial stem cells, obtained from any suitable source.

The patch may be adapted to each patient by using autologous or allogeneic stem cells or an engineered stem cell line that may be suitable to all patients. The patch may also be potentially used for patients that have compromised function of stem cells, such as in elderly patients or patients that suffer from diseases such as osteoporosis etc.

Any of the patches described herein above may be used for screening and/or toxicity studies to identify therapeutically effective agents that promote and/or modulate the endogenous regeneration and/or repair of biological soft tissue, connective tissue or bone.

The invention also encompasses a method of manufacturing a biological tissue-regeneration patch as described herein above. The method comprises: i) preparing a film from a biocompatible polymer; ii) functionalising the surface of the polymer; and iii) conjugating one or more proteins from the Wnt family, or an agonist of the Wnt signalling pathway, to the polymer surface.

As described above, the surface of the polymer may be functionalised to provide primary amine functional groups. One example of such functionalisation is by treatment of the surface with oxygen plasma and aminopropyl-triethoxysilane (APTES), although other suitable treatments are well known to the skilled person and are encompassed by the scope of this disclosure.

The method may further comprise culturing stem cells after conjugation of Wnt, an agonist thereof, or an Rspo protein, onto the functionalised polymer surface. Ideally, the stem cells are cultured as a monolayer. A culture time of between 5 and 10 days, preferably 7 to 8 days has been found to be ideal. After this time, a 3D structure has been formed.

The present invention also encompasses a biological tissue-regeneration method, comprising implanting the patch as described hereinabove across or over a defect, hole, niche, tear or fracture.

Thus, the Wnt-bandage of the present invention acts on the bone, organ or tissue of a patient. When the fracture or tissue heals, the bandage may be removed, or a biodegradable bandage may be used to avoid removal.

It will be appreciated that the Wnt bandage of the present invention may be used to promote endogenous healing. Although the present invention is exemplified with respect to bone repair and osteogenesis, the Wnt-bandage may be used to engineer niches of other organs because almost all stem cells are Wnt-responsive and rely on Wnt for self-renewal. These tissue model-bandages may be used to repair tissues of the damaged organ or/and fulfil functions of the damaged organ. Additionally, the tissue models may be used for drug screening and toxicity studies.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The term also encompasses “consisting of” and “consisting essentially of”.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the term “one or more”, such as one or more members of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Wnt3a

It will be appreciated that the examples herein describe production of mouse Wnt3a according to the method of Willert et al 2003, and production of patches (bandages) comprising same.

In some embodiments it may be desirable to use the human Wnt3a, and suitably production of patches (bandages) described herein comprising same human Wnt3a. Thus, suitably in this embodiment Wnt3a means a polypeptide having the amino acid sequence of human Wnt3a, such as UniProt accession number P56704 (SEQ ID NO: 1):

```
                10 20 30 40
MAPLGYFLLL CSLKQALGSY PIWWSLAVGP QYSSLGSQPI
                50 60 70 80
LCASIPGLVP KQLRFCRNYV EIMPSVAEGI KIGIQECQHQ
                90 100 110 120
FRGRRWNCTT VHDSLAIFGP VLDKATRESA FVHAIASAGV
              130 140 150 160
AFAVTRSCAE GTAAICGCSS RHQGSPGKGW KWGGCSEDIE
              170 180 190 200
FGGMVSREFA DARENRPDAR SAMNRHNNEA GRQAIASHMH
              210 220 230 240
LKCKCHGLSG SCEVKTCWWS QPDFRAIGDF LKDKYDSASE
              250 260 270 280
MVVEKHRESR GWVETLRPRY TYFKVPTERD LVYYEASPNF
              290 300 310 320
CEPNPETGSF GTRDRTCNVS SHGIDGCDLL CCGRGHNARA
              330 340 350
ERRREKCRCV FHWCCYVSCQ ECTRVYDVHT CK
```

Suitably human Wnt3a is prepared exactly as described for mouse Wnt3a as is well known in the art using the method of Willert et al (Willert et al (2003) Nature 22; 423(6938): 448-52), the only difference being the use of nucleotide sequence encoding the human Wnt3a amino acid sequence in place of the mouse sequence used by Willert et al.

A suitable nucleic acid coding sequence can be derived from the above amino acid sequence using the universal genetic code.

In case any further guidance is needed, an exemplary human coding sequence is provided below—GenBank Accession Number AB060284 (SEQ ID NO: 2):

>ENA|AB060284|AB060284.1 Homo sapiens mRNA for WNT3A,
complete cds.
CGGCGATGGCCCCACTCGGATACTTCTTACTCCTCTGCAGCCTGAAGCAGGCTCTGGGCA
GCTACCCGATCTGGTGGTCGCTGGCTGTTGGGCCACAGTATTCCTCCCTGGGCTCGCAGC
CCATCCTGTGTGCCAGCATCCCGGGCCTGGTCCCCAAGCAGCTCCGCTTCTGCAGGAACT
ACGTGGAGATCATGCCCAGCGTGGCCGAGGGCATCAAGATTGGCATCCAGGAGTGCCAGC
ACCAGTTCCGCGGCCGCCGGTGGAACTGCACCACCGTCCACGACAGCCTGGCCATCTTCG
GGCCCGTGCTGGACAAAGCTACCAGGGAGTCGGCCTTTGTCCACGCCATTGCCTCAGCCG
GTGTGGCCTTTGCAGTGACACGCTCATGTGCAGAAGGCACGGCCGCCATCTGTGGCTGCA
GCAGCCGCCACCAGGGCTCACCAGGCAAGGGCTGGAAGTGGGGTGGCTGTAGCGAGGACA
TCGAGTTTGGTGGGATGGTGTCTCGGGAGTTCGCCGACGCCCGGGAGAACCGGCCAGATG
CCCGCTCAGCCATGAACCGCCACAACAACGAGGCTGGGCGCCAGGCCATCGCCAGCCACA
TGCACCTCAAGTGCAAGTGCCACGGGCTGTCGGGCAGCTGCGAGGTGAAGACATGCTGGT
GGTCGCAACCCGACTTCCGCGCCATCGGTGACTTCCTCAAGGACAAGTACGACAGCGCCT
CGGAGATGGTGGTGGAGAAGCACCGGGAGTCCCGCGGCTGGGTGGAGACCCTGCGGCCGC
GCTACACCTACTTCAAGGTGCCCACGGAGCGCGACCTGGTCTACTACGAGGCCTCGCCCA
ACTTCTGCGAGCCCAACCCTGAGACGGGCTCCTTCGGCACGCGCGACCGCACCTGCAACG
TCAGCTCGCACGGCATCGACGGCTGCGACCTGCTGTGCTGCGGCCGCGGCCACAACGCGC
GAGCGGAGCGGCGCCGGGAGAAGTGCCGCTGCGTGTTCCACTGGTGCTGCTACGTCAGCT
GCCAGGAGTGCACGCGCGTCTACGACGTGCACACCTGCAAGTAGGCACCGGCCGCGGCTC
CCCCTGGACGGGGCGGGCCCTGCCTGAGGGTGGGCTTTTCCCTGGGTGGAGCAGGACTCC
CACCTAAACGGGGCAGTACTCCTCCCTGGGGGCGGGACTCCTCCCTGGGGGTGGGGCTCC
TACCTGGGGGCAGAACTCCTACCTGAAGGCAGGGCTCCTCCCTGGAGCTAGTGTCTCCTC
TCTGGTGGCTGGGCTGCTCCTGAATGAGGCGGAGCTCCAGGATGGGGAGGGGCTCTGCGT
TGGCTTCTCCCTGGGGACGGGGCTCCCCTGGACAGAGGCGGGGCTACAGATTGGGCGGGG
CTTCTCTTGGGTGGGACAGGGCTTCTCCTGCGGGGGCGAGGCCCCTCCCAGTAAGGGCGT
GGCTCTGGGTGGGCGGGGCACTAGGTAGGCTTCTACCTGCAGGCGGGGCTCCTCCTGAAG
GAGGCGGGGCTCTAGGATGGGGCACGGCTCTGGGGTAGGCTGCTCCCTGAGGGCG

When particular amino acid residues are referred to herein using numeric addresses, the numbering is taken with reference to the wild type Wnt3a amino acid sequence (or to the polynucleotide sequence encoding same if referring to nucleic acid). Exemplary sequences have been provided above.

Suitably the current version of sequence database(s) is relied upon. Alternatively, the release in force at the date of filing is relied upon. For the avoidance of doubt, UniProt release 2019_11 is relied upon. In more detail, the UniProt consortium European Bioinformatics Institute (EBI), SIB Swiss Institute of Bioinformatics and Protein Information Resource (PIR)'s UniProt Knowledgebase (UniProtKB) Release 2019_11 is relied upon. UniProt (Universal Protein Resource) is a comprehensive catalogue of information on proteins (“UniProt: the universal protein knowledgebase” Nucleic Acids Res. 45: D158-D169 (2017)).

GenBank® is the NIH genetic sequence database, an annotated collection of all publicly available DNA sequences (Nucleic Acids Research, 2013 January; 41(D1): D36-42). GenBank is part of the International Nucleotide Sequence Database Collaboration, which comprises the DNA DataBank of Japan (DDBJ), the European Nucleotide Archive (ENA), and GenBank at NCBI. For the avoidance of doubt, GenBank release 234 of Oct. 15, 2019 is relied upon.

This is to be used as is well understood in the art to locate the residue of interest. This is not always a strict counting exercise—attention must be paid to the context. For example, if the protein of interest is of a slightly different length, then location of the correct residue in that sequence may require the sequences to be aligned and the equivalent or corresponding residue picked. This is well within the ambit of the skilled reader.

Mutating has its normal meaning in the art and may refer to the substitution or truncation or deletion of one or more residues, motifs or domains. Mutation may be effected at the polypeptide level, for example, by synthesis of a polypeptide having the mutated sequence, or may be effected at the nucleotide level, for example, by making a polynucleotide encoding the mutated sequence, which polynucleotide may be subsequently translated to produce the mutated polypeptide.

In the following experiment, the inventors initially further validate the cellular identity of the WIOTM by using the recently identified stem cell marker Cadherin 13 (CDH13) (Holley, R. J. et al (2015) Stem Cell Reports 4: 473-488), and the differentiation marker Osteopontin (OPN). It was found that the expression pattern of CDH13 and OPN in the WIOTM are similar to Stro1 and OCN, respectively. Next, the molecular mechanism that underlies the formation of the WIOTM was dissected. The inventors also evaluate the regenerative capacity of localised Wnt3a and the WIOTM in in vivo murine model of critical size bone defects of the calvarial bone.

Wnts were covalently immobilised to materials and a human Wnt-induced osteogenic tissue model (WIOTM) was engineered that maintains a human skeletal stem cell (hSSC) population and a cascade of differentiating osteogenic cells in 3D within one week. The results show that Wnt-mediated asymmetric stem cell division drives this process. In dividing hSSCs, localised Wnt proteins (Wnts) polarise components of the Wnt/β-catenin pathway, polarity proteins, and the stem cell marker Cadherin-13 close to the Wnt source. The spindle is oriented perpendicular to localised Wnts, which also polarise differentiation markers farther from the Wnt, leading to oriented asymmetric cell division.

To probe the regenerative capacity of localised Wnts and the WIOTM in vivo, Wnts were covalently tethered to a biodegradable film (Wnt-bandage) and the WIOTM was generated on the bandage. Each bandage was overlaid onto critical size calvarial bone defects in immunocompromised mice. After eight weeks, the Wnt-bandage had significant endogenous repair in comparison to controls. The WIOTM-bandage further improved the repair by generating additional new bone tissues. Importantly, Wnt-responsive hSSCs were found close to the Wnt-bandage and formation of mature new bone cells that are descendants of hSSCs.

Thus, the results provide a molecular understanding of human osteogenesis and a novel approach to repair bone defects.

The present invention will now be described by way of example only with reference to the following non-limiting embodiments.

### Examples

Materials and Methods

Culture of Human Skeletal Stem Cells

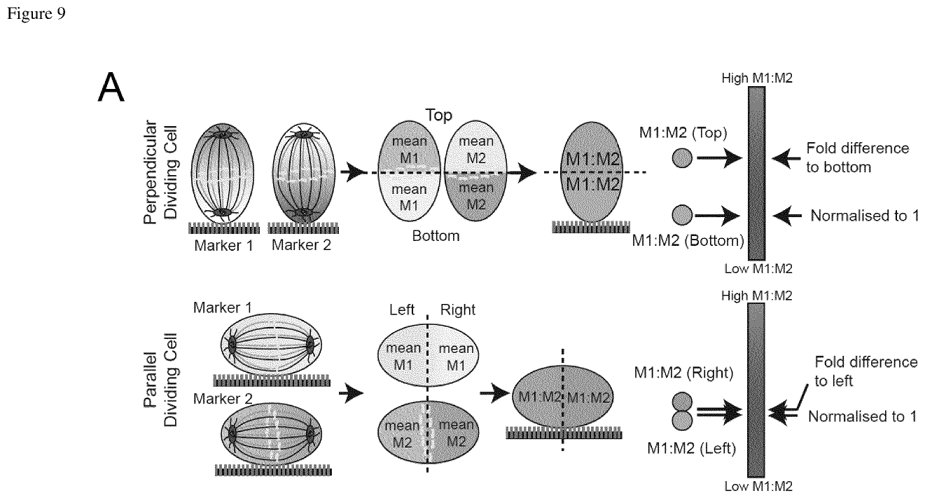

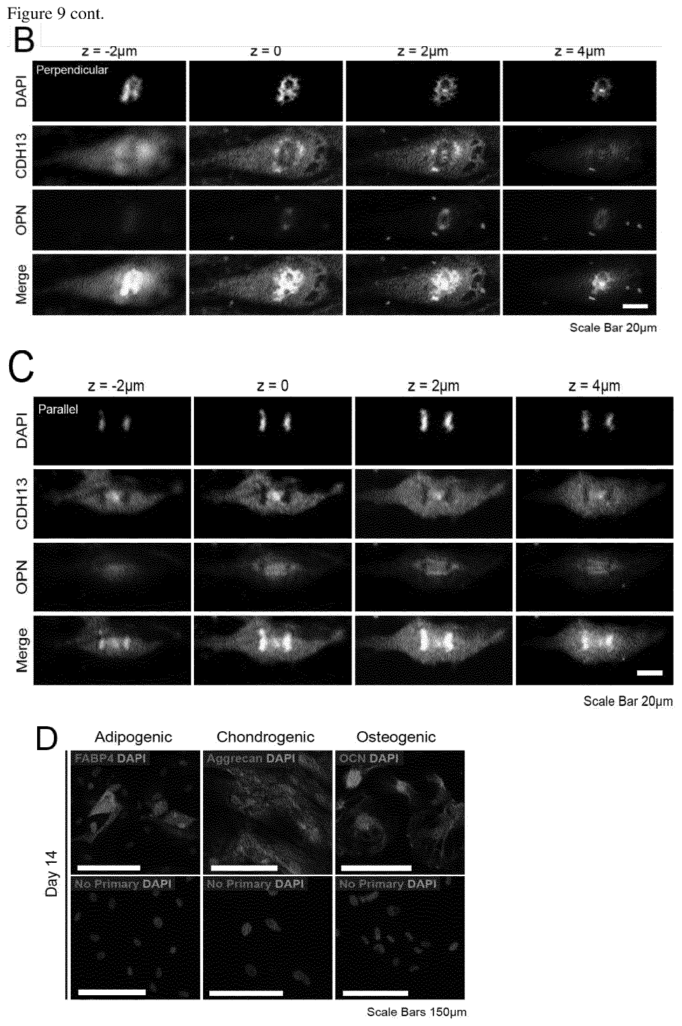

Human mesenchymal stem cells (hSSCs) were isolated from a commercially available bone marrow aspirate (AllCells LLC). The bone marrow aspirate was plated onto culture flasks pre-coated with 10 ng/ml fibronectin (Sigma-Aldrich, UK) and cells allowed to attach. Characterisation for surface marker expression (CD73+, CD90+, CD105+, and CD45−, CD34−, CD14−, CD19−, and HLA-DR−) was then carried out as previously described (Lowndes et al (2016) supra; Marshak et al (1999)). hSSCs were expanded up to passage 5 (P5) in basal media: high glucose (4.5 g/L) Dulbecco's Modified Eagle Media (DMEM) supplemented with 10% foetal bovine serum (FBS), 1% L-glutamine (Lonza), and 1% Penicillin/Streptomycin (Lonza) at 37° C. in humidified air with 5% CO₂. Multipotency was confirmed at P5 by differentiation into adipogenic, chondrogenic and osteogenic lineages as determined by immunofluorescence (R&D Systems, SC006; FIG. 9D). hSSCs stably infected with 7TCF-eGFP/SV40-mCherry (Fuerer, C. & Nusse, R. (2010) PLoS ONE 5: e9370.) were also used for some experiments. These cell lines were cultured and passaged under standard conditions.

WNT3A Purification

Recombinant mouse Wnt3A was produced in Drosophila S2 cells grown in suspension culture and purified by Blue Sepharose affinity and gel filtration chromatography as described (Willert et al (2003) Nature 22; 423(6938): 448-52). Wnt3A activity was determined in a luciferase reporter assay using L cells stably transfected with the SuperTOPFlash reporter as described (Mikels A. J. & Nusse R. (2006) PLoS Biol. 4(4):el 15).

Functionalisation of Wnt Platform

To functionalise the Wnt platform (Corning PureCoat Amine 96-well flat-bottom multiwell plates, VWR International, cat. no. 734-1475), amine-treated wells in the plate were washed once with 100 μl of Dulbecco's phosphate buffer saline (dPBS) and then incubated with 50 μl of 5% (v/v) glutaraldehyde (Sigma-Aldrich) for 30 minutes at room temperature in the dark. The wells were washed three times with 100 μl of dPBS, and the last wash was left to incubate for 10 minutes. 60 ng of Wnt3A in PBS was incubated on the wells for 1 hour at room temperature. The wells were also washed three times with 100 μl of dPBS, and the last wash was left to incubate for 10 minutes. For inactive Wnt platforms, 100 μl of 20 mM DTT was incubated on the wells for 30 minutes at 37° C. The wells were washed three times with 100 μl of dPBS. After removing the final wash, the wells were incubated with the hSSCs basal media for at least 1 hour.

Culture of Human Skeletal Stem Cells on Wnt Platform

Cells were seeded onto functionalised wells at 2,500 cells/well with 100 μl of hSSCs basal media and cultured for 24 hours, after which the media was removed and 40 μl of 1 mg/ml rat tail collagen 1 (Corning) was laid over the cell monolayer. Before adding, rat tail collagen 1 was neutralised with 1M NaOH (23 μl per 1 ml of original collagen gel; 0.3% v/v) and diluted in serum-free media. The wells were incubated for 2 hours at 37° C. for introducing gel crosslinking. 150 μl of osteogenic media was then topped up on the gel. Osteogenic media was composed of basal medium with the addition of dexamethasone (0.1 μM), β-glycerophosphate (10 mM), ascorbic acid (50 μM) and non-essential amino acids (1% v/v). Samples were cultured for three days at 37° C. in humidified air with 5% CO₂, with media changed every 2 days.

Immunofluorescent Staining of Wnt Platform

After one to seven days incubation, osteogenic media was removed, the wells were washed once with 100 μl of dPBS and fixed with 4% paraformaldehyde (PFA) for 30 minutes. The wells were then washed four times with 1% bovine serum albumin (BSA) in dPBS (1% BSA/PBS) for 30 minutes each. The samples were permeabilised with 100 μl of 0.25% Triton X-100 in 1% BSA/dPBS for 30 minutes. The wells were again washed four times with 1% BSA/PBS for 30 minutes each. 10% goat serum in 1% BSA/dPBS was added to the samples for 2 hours at room temperature. 100 μl of primary antibody cocktails was then added to the samples, which was incubated overnight at room temperature. Primary antibody cocktails in 1% BSA/dPBS are the following: β-Catenin (BD Transduction Laboratories, 610154) (1:250) and APC (Santa Cruz, SC-7930) (1:250); Stro1 (R&D Systems, MAB1038) (1:50) and Osteocalcin (OCN) (Abcam, ab93876) (1:500); Cadherin H (CDH13) (Abcam, ab36905) (1:100) or Plexin A2 (PLXNA2) (Abcam, ab39357) and Osteopontin (OPN) (Santa Cruz, sc21742) (1:100); Centrin 1 (04-1624) (1:150) and Ninein (Abcam, ab4447) (1:500), NUMB (Abcam, ab4147) (1:250), aPKCζ (Santa Cruz, sc17781), Tubulin (Abcam, ab6160) (1:1000). The wells were washed four times with 1% BSA/PBS for 30 minutes each. The samples were then incubated with secondary antibody (1:1000) and 4′,6-diamidino-2-phenylindole (DAPI) (1:1000) (ThermoFisher) cocktails for 2 hours at room temperature or overnight at 4° C. The secondary antibodies used were the following: donkey anti-mouse IgG AF488 (Thermofisher, A21202), donkey anti-Rabbit IgG AF647 (Thermofisher, A32795), donkey anti-goat IgG AF555 (Thermofisher, A32816), donkey anti-rat IgG AF488 (Thermofisher, A21208), donkey anti-mouse IgG AF647 (Thermofisher, A31571), goat anti-mouse IgM AF488 (ThermoFisher, A21042), goat anti-rabbit IgG AF555 (ThermoFisher, A21428), goat anti-rabbit IgG AF488 (ThermoFisher, A11034) and goat anti-mouse IgG AF555 (ThermoFisher, A21424). After a two-hour incubation, the wells were washed four times with 1% BSA/PBS for 30 minutes each. The samples were stored in 0.05% w/v sodium azide in dPBS at 4° C. until their images were taken.

Imaging and Analysis of Wnt Platform

To analyse the distribution of the components of Wnt/β-catenin pathway and centrosome, images with a variety of thickness were taken using Zeiss microscope, Inverted Spinning Disk Confocal microscope (Nikon) and Leica Confocal microscope (Leica SP8) depending on the imaging depth required. Step size was determined according to the Nyquist criteria for sampling, with total thickness <200 μm. All the imaging data were analysed using ImageJ software. In perpendicular dividing cell analysis, after making the targeted cell boundary as an ROI (region of interest), the plane with maximum intensity DAPI was taken as the centre, indicating the mitotic plane. Mean intensity of the protein markers were measured either side of this plane, followed by comparison and normalisation to calculate a fold difference. In parallel dividing cell analysis, two ROIs were set up within the dividing cell depending on the cell dividing direction. For divided cells, the demarcation between cells was taken as the cell borders, or if not clear, the centre point between DAPI signals.

Fabrication of Biodegradable Polymers

Polycaprolactone (PCL) or poly(lactic-co-glycolic acid) (PLGA) (Sigma-Aldrich, UK) films with a mass of 0.3 g were solvent cast in 7 cm glass dishes at a concentration of 2% (w/v) in anisole (Sigma-Aldrich) and left for 72 hours at 50° C. to allow the solvent to evaporate.

Glutaraldehyde Group Introduction to Biodegradable Polymers

After 72 hours, the films were carefully removed from the dishes and treated with 2% (w/v) hexamethylenediamine (HMIDA) (Sigma-Aldrich) in 90% (v/v) isopropanol (Sigma-Aldrich) for 1.5 hours on a tube roller at room temperature to introduce amine groups to the surface of the films. The films were then washed in 100% isopropanol three times for 10 minutes each and left to dry. For plasma-chemical activation, films were plasma activated via plasma etching electrode in oxygen gas flow of 20 sccm, radio frequency plasma generator (frequency 13.56 MHz, power 50 W Diener electronic Zepto-W6, Ebhausen, Germany) at 0.2 mbar pressure for 3 minutes. The freshly O₂ plasma activated films were treated with 5% 3-aminopropyltriethoxysilane (APTES) (Sigma-Aldrich) in ethanol (Sigma-Aldrich) immediately for 2 hours. The films were then washed in 100% ethanol three times for 10 minutes each and left to dry. These amine-group reacted films can keep their activity by being stored at 4° C. for up to three months. The amine groups were further reacted with 0.05% (w/v) glutaraldehyde (Sigma-Aldrich) in 70% (v/v) ethanol (Fisher Scientific) for 5 minutes at room temperature to introduce aldehyde groups on to the surface of the polymer. The polymers were then washed three times with 100% ethanol and left to dry. The polymers were sterilised in 100% ethanol for 15 minutes before being washed three times in dPBS.

Wnt3a Modification of Biodegradable Polymers

Purified Wnt3a solution prepared as above mentioned was then diluted in dPBS for a final volume of 20 μl per drop to cover a working area of −29 mm² on 6 mm films with amounts of 20 ng or 60 ng of Wnt3A. The films were incubated with soluble Wnt3a for 1 hour and washed three times with PBS, and then further incubated with high glucose DMEM containing 20% FBS to block any unreacted aldehyde groups. BSA and inactivated Wnt3a films were also produced as a control. This was achieved by either reacting the aldehyde groups with 0.1% (w/v) BSA for 1 hour at room temperature instead of using the Wnt3A and by inactivating the Wnt3A modified polymers with 20 μl of 20 mM DDT for 30 minutes at 37° C. respectively.

Culture of Human Skeletal Stem Cells (hSSCs) on Wnt Functionalised and Control Biodegradable Polymers

For in vitro analysis of functionalised films, hSSCs with the 7×TCF-eGFP/SV40-mCherry reporter were seeded onto functionalised polymer films at 35,000 cells/cm² (10,000 cells/bandage) and cultured in hSSCs basal media for 24 hours. For control experiments, hSSCs were seeded onto 96-well plates at the same dilution and cultured in the presence of either 50 ng/ml or 150 ng/ml soluble Wnt3A or 0.1% BSA solution for 24 hours.

For the preparation of in vivo cellular bandages, functionalised polymer films (active Wnt3a in WIOTM-bandages, inactive Wnt3a in iWnt-hSSC-bandages) were seeded with cells at the same density and allowed to adhere. The basal media was then removed and 100 μl of 1 mg/ml rat tail collagen 1 was overlaid, allowed to set, and topped up with osteogenic media, as detailed above. WIOTM- and iWnt-hSSC bandages were cultured for 7 and 2 days respectively, with three and two media changes respectively.

Analysis of Wnt Activity on Wnt-Functionalised Biodegradable Polymers

Fluorescent intensity from Wnt-responsive hSSCs stably infected with 7×TCF-eGFP/SV40-mCherry was measured using Operetta High-Content Imaging System (PerkinElmer). Before imaging, cells were stained with Hoechst33342 (ThermoFisher) for 5 minutes, washed with dPBS, and then media refreshed with 20 μl of dPBS containing 10% FBS. L cells transfected with SuperTOPFlash reporter (LS/L) were cultured on Wnt-activated surfaces in DMEM with 10% FBS and 1% penicillin/streptomycin. The Wnt activity of functionalised biodegradable polymers was also measured by culturing the L cells transfected with SuperTOPFlash reporter (LS/L) in DMEM supplemented with 10% FBS and 1% penicillin/streptomycin overnight and quantified the Wnt-induced luciferase activity using Dual-Light Systems (Applied Biosystems).

Western Blot Analysis of Wnt Functionalised Biodegradable Polymers

To determine protein levels in the input and washes during film surface functionalisation, protein electrophoresis and western blotting was used. After Wnt3A modification of the polymer films, Wnt3A or control solution was collected for western blot (WB), and films were washed in 20 μl of dPBS, which was also collected for WB three times. Samples were mixed with 4× Laemmli buffer (Bio-Rad) and loaded onto Mini-PROTEAN TGX stain-free gels (Bio-Rad) and then immunoblotted for αWNT3A (Millipore; 09-162) overnight at 4° C. in 5% milk in TBST (Tris-buffered saline and Tween 20; 1:1,000) after 1 hour blocking in 5% milk in TBST. Bands were visualised with chemiluminescence on the ChemiDoc (Bio-Rad).

Preparation of Wnt- or Wnt-Induced Human Osteogenic Tissue Model (WIOTM)-Bandage

Wnt3A-modified PCL polymers used in transplantation experiments are named Wnt-bandage. Wnt-bandages were prepared on the day before the transplantation surgery. On the other hand, Wnt3a-modified PCL polymers on which hSSCs were cultured for one week under 3D culture condition as mentioned in Wnt platform experiment are named WIOTM-bandage. In WIOTM-bandages, polymers were prepared from 8 days before the surgery. After 24-hours incubation of Wnt-bandage with the cells prepared as above mentioned, the basal media was removed, and 100 μl of 1 mg/ml rat tail collagen 1 was laid over the cell monolayer. Before adding, rat tail collagen 1 was prepared as above mentioned. The wells were incubated for 2 hours at 37° C. for introducing gel crosslinking. 150 μl of osteogenic media were then topped up on the gel. Osteogenic media were changed 1 every two to three 2 days and cultured for seven days before the surgery.

Transplantation of Wnt Functionalised Biodegradable Polymers with or without hSSCs

Thirteen-week-old female severe combined immunodeficient (SCID) mice were used for transplantation of Wnt-bandage and WIOTM-bandage into calvarial defects. All procedures were carried out under a sterile condition. Firstly, general anaesthesia was administered into mice via the intraperitoneal route using an anaesthetic cocktail (10 μl/body weight), which consisted of sterile sodium chloride 0.9% saline with 7.5 mg/ml of Ketamine (Vetalar®) and 0.1 mg/mL of Medetomidine (Domitor®). After testing the loss of tail and hind paw withdrawal reflex, the top of the head was shaved with a mini hair trimmer and lidocaine 5% M/M Ointment® was spread to promote local anaesthesia. Eyes were lubricated with Viscotears® Liquid gel and a sagittal skin incision was made along the midline with a scalpel. Mice were put under a stereoscope microscope. The cranial vault was exposed and the periosteal layer overlying the skull was removed with an outward separating motion using the cotton buds. One or two 4 mm defects were drilled gently using a dental handpiece, avoiding the build-up of heat by friction. The bone pieces were removed and the defects covered with Wnt-bandage or WIOTM-bandage with 40 μl of 1 mg/ml rat tail collagen 1 and fixed in place using Vetbond® before the skin incision was also closed using Vetbond®. A recovery cocktail consisting of sterile sodium chloride 0.9% saline with 1.25 mg/ml of atipamezole (Antisedan®) and 0.01 mg/mL of buprenorphine (Vetergesic®) was injected to mice (10 μl/body weight) which were observed very carefully in the 28° C. incubator.

microCT Scanning and Analysis

8 weeks after the transplantation, mice head samples were harvested. Whole head samples were fixed with 40 mL of 4% PFA in a plastic tube on a rotor at room temperature overnight. The samples were washed three times with PBS for 15 minutes each next day. The head samples were then scanned using a Scanco pCT50 microCT scanner (Scanco, Bruttisellen, Switzerland). The specimens were immobilised in 19 mm scanning tubes using cotton gauze and scanned to produce voxel size volumes of side length 10 μm, using X-ray settings of 70kVp, 114 μA and a 0.5 mm aluminium filter to attenuate harder X-rays. The scans were automatically scaled at reconstruction using the calibration object provided by the CT manufacturer, consisting of five rods of hydroxyapatite (HA) at concentrations of 0 to 790 mg HA/cm³, and the absorption values expressed in Hounsfield Units (HU). The specimens were characterised using Parallax Microview software package and 3D images were reconstructed from the raw CT scanning data. The images were analysed for area and volume quantification of new bone regeneration (Parallax Innovations Inc., Ilderton, ON Canada). Background signal corresponding to soft non-mineralized tissue and air (<250 mg HA/cm³) was removed by threshold, and mineralised tissue in the defect was considered as a density >500 mg HA/cm³.

Cryosection Preparation

To trim the samples, the head skin and brain were removed from a fixed mouse head with scissors and two sets of tweezers. After the trimming, the samples were put into 10% formic acid overnight for decalcification. Dehydration was carried out over two nights, firstly with 30% sucrose in water, then 30% sucrose and 30% Optimal Cutting Temperature (OCT) compound in water. Samples were flash frozen in cryomolds filled with OCT compound, cooled by dry ice and 100% ethanol. Mice head samples were transversely sectioned in the anterior to posterior direction using Cryostats® at a 12-20 μm slice. Whole samples were stored at −80° C. until they were sectioned. Sectioned slices were stored at −20° C. until they were stained.

Histological Staining of Tissue Sections

Haematoxylin (Vector Labs., H-3404) and Eosin (Sigma-Aldrich, 318906) (H&E) staining was performed by standard protocol. Movat's Pentachrome staining (Abcam, ab245884) was performed according to the supplied method. Sections were mounted under a coverslip with DPX (Sigma-Aldrich, 06522).

Immunofluorescence (IF) and Immunohistochemistry (IHC) Staining of Tissue Sections Firstly,

OCT compound was removed from the sectioned slides by putting the samples into pre-warmed water (37° C.) for 15 minutes each. IHC sections were blocked with supplied hydrogen peroxide block as part of the HRP/AEC detection IHC kit (Abcam, ab93705). IF sections were washed and blocked three times using PBST (0.1% Triton X-100, 1% BSA in PBS) for 15 minutes each. The slides were then incubated with primary antibodies diluted in staining buffer (IF: 0.05% TritonX-100, 1% BSA in PBS, IHC: PBS) as listed above, with the addition of: 1:250 human O₂-Microglobulin (h3MG2) (Sigma-Aldrich, M7398), 1:100 Sclerostin (Abcam, ab85799 and Bio-Rad, HCA230Z), 1:400 GFP (Aves Lab, GFP−1020), and 1:400 RFP (Rockland, 600-401-379) overnight at 4° C. For IF, PBST was used to wash the slides before incubation with the secondary antibodies for 1 hour at room temperature. The secondary antibodies used were goat anti-mouse IgM AF488, goat anti-rabbit IgG AF555 (ThermoFisher, A21428), goat anti-rabbit IgG AF488 (ThermoFisher, A11034), and goat anti-mouse IgG AF555 (ThermoFisher, A21424), as described above, with the addition of donkey anti-Chicken IgY AF488 (Jackson ImmunoResearch Labs., 703-545-155). IF sections were then washed three times using PBST and counterstained using 1:1000 DAPI. For IHC, the slides were further treated as described in the IHC kit and counterstained with Haematoxylin (Vector Labs., H-3404). Slides were sealed with aqueous mounting media (Abcam, ab128982) prior to imaging. All types of staining were confirmed in tissues of at least three separate animals.

In Vivo Quantification

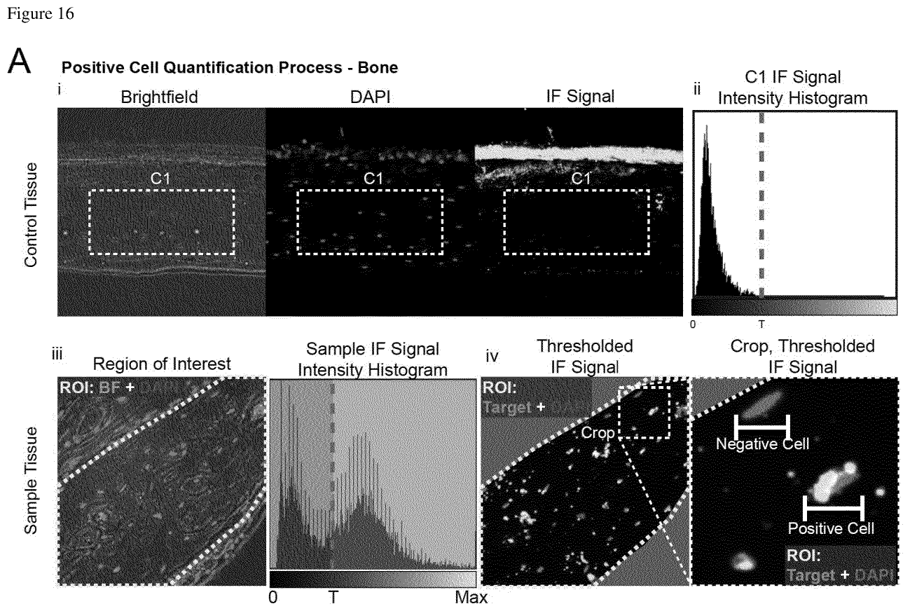

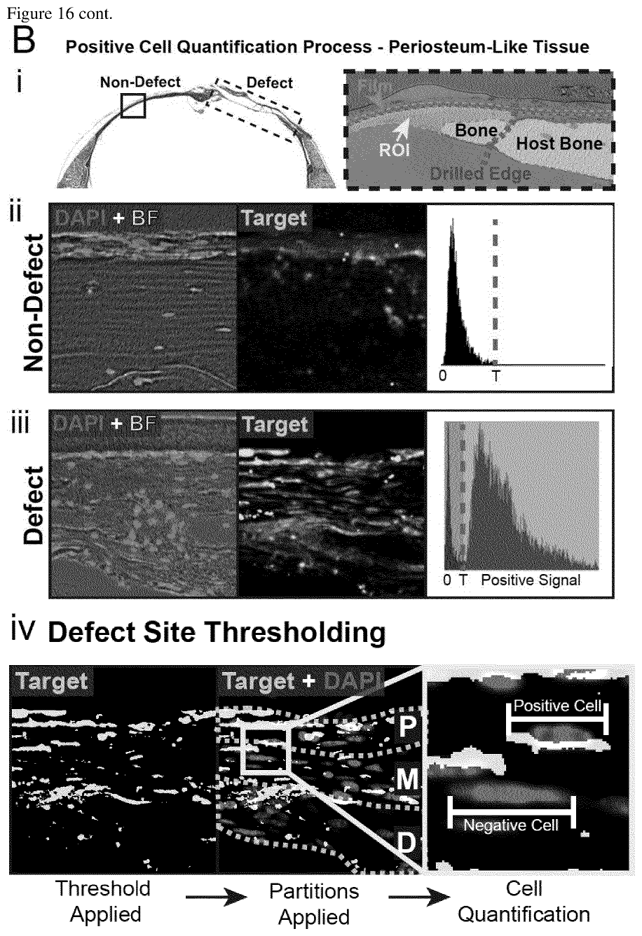

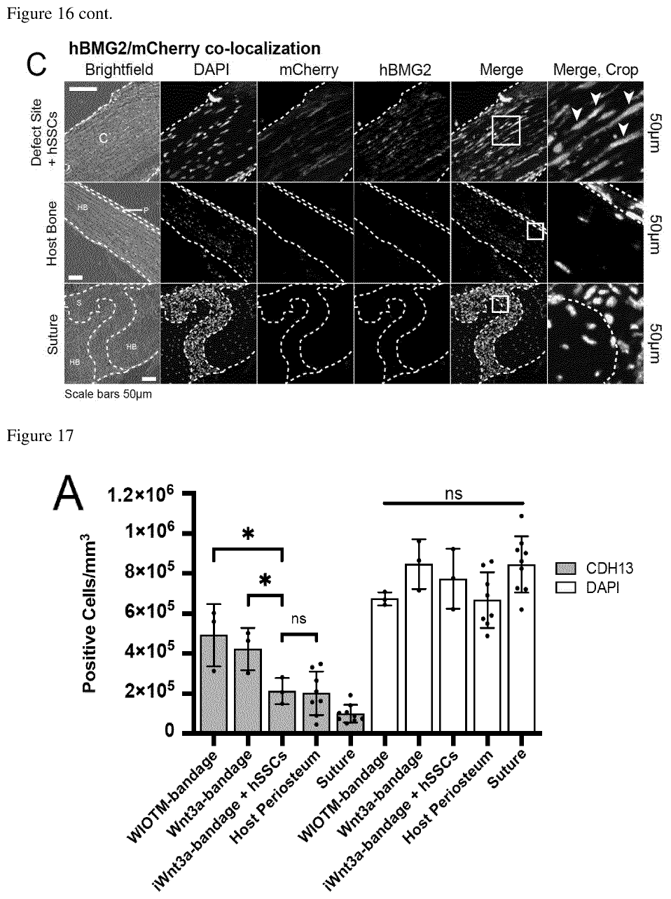

The quantification of positive cells was determined by a threshold method of immunofluorescence images, as described in FIG. 16A-B. An area of stained control tissue was selected, and the highest intensity cellular signal for the marker of interest was determined. This intensity was used as the threshold for the sample tissue, such that all remaining signal was of a higher intensity than the control tissue. Positive cells could then be quantified by the presence of cellular signal.

Results

Localised Wnt3a- and WIOTM-bandages were engineered and their ability to repair severe calvarial defects was evaluated in vivo.

Localised Wnt3a Proteins Oriented the Spindle of hSSCs to Generate a 3D Wnt-Induced Human Osteogenic Tissue Model (WIOTM)

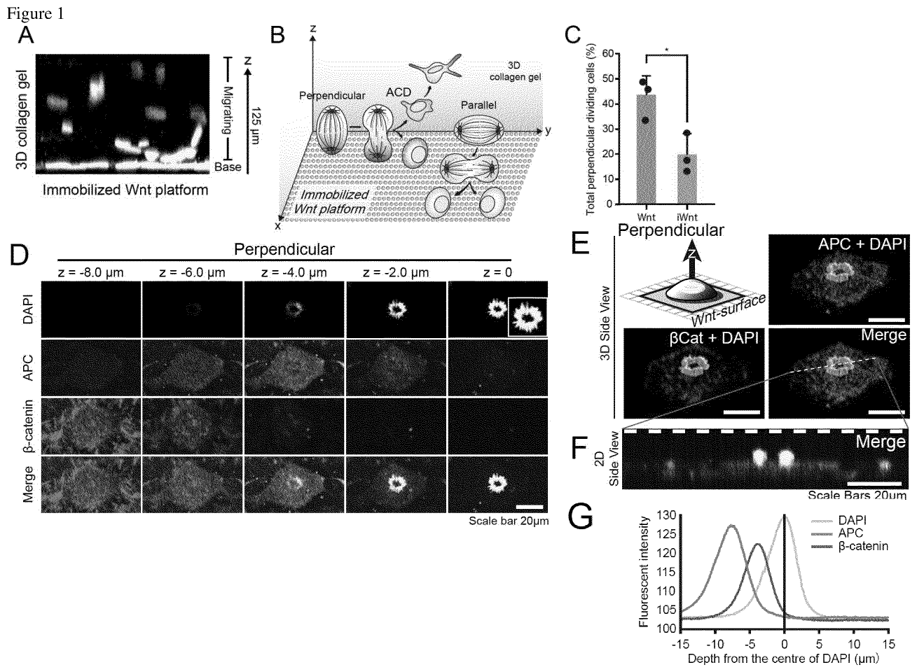

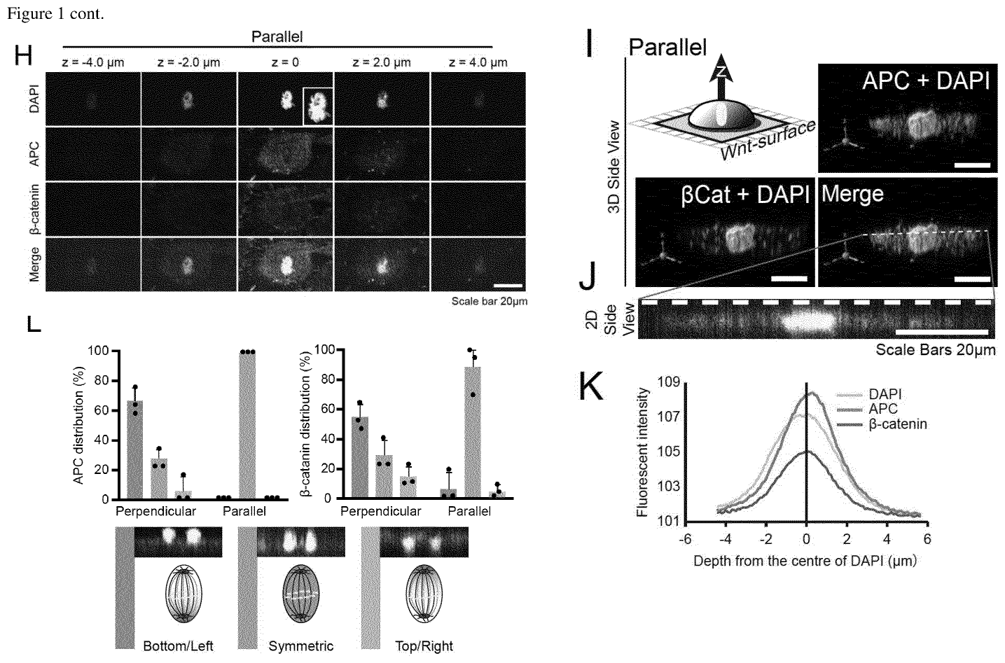

In a previous study (Lowndes, M. et al (2017) supra), it has been shown that immobilised Wnt3a platforms can maintain hSSCs close to the Wnt3a source and generate a cascade of increasingly differentiating osteogenic cells in 3D collagen type1 gel to form the WIOTM (FIG. 1A). In this experiment, understanding the underlying molecular mechanism of this process was of interest. Unlike immobilised Wnt3a, soluble Wnt3a proteins added to the media can induce the proliferation of hSSCs but did not promote cell migration (Lowndes, M. et al (2017) supra). Thus, it was speculated that the orientation of cell division and multilayer formation are coupled and regulated by the asymmetric presentation of Wnt3a to one side of the hSSCs (see FIG. 1B).

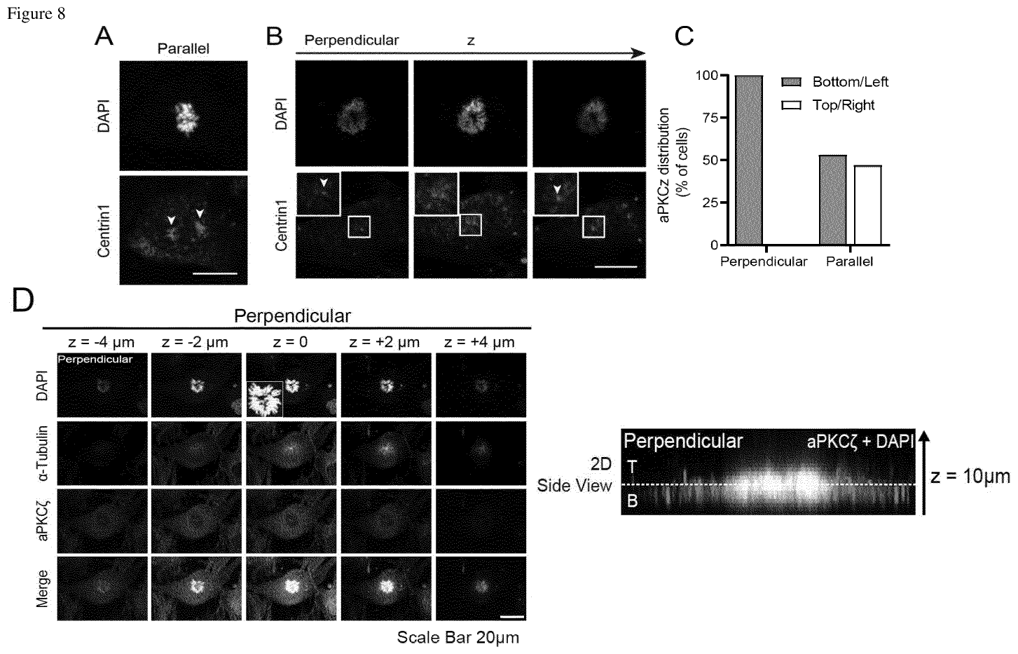

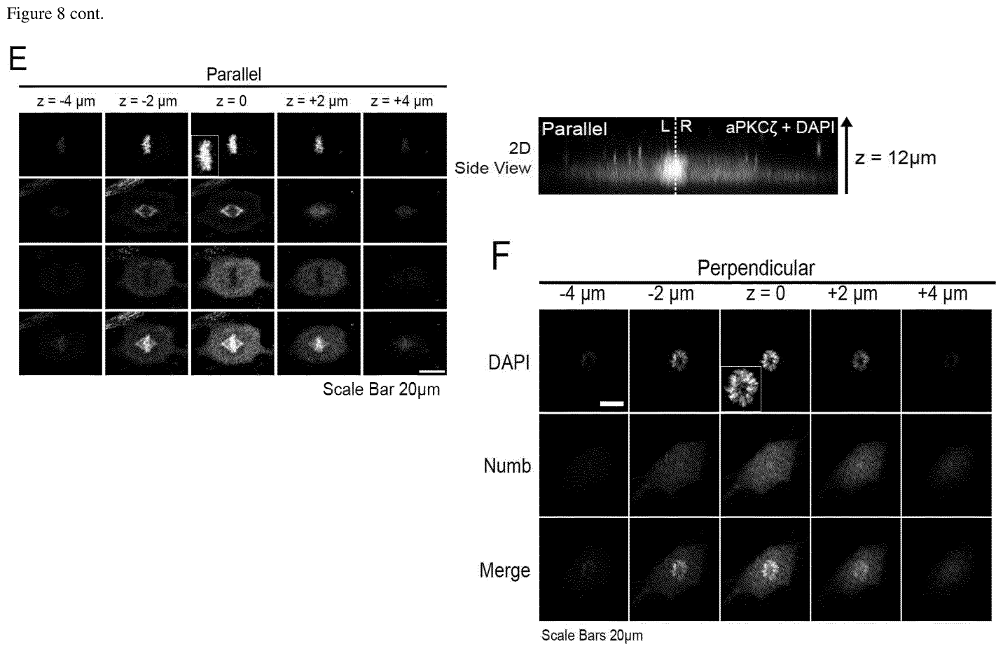

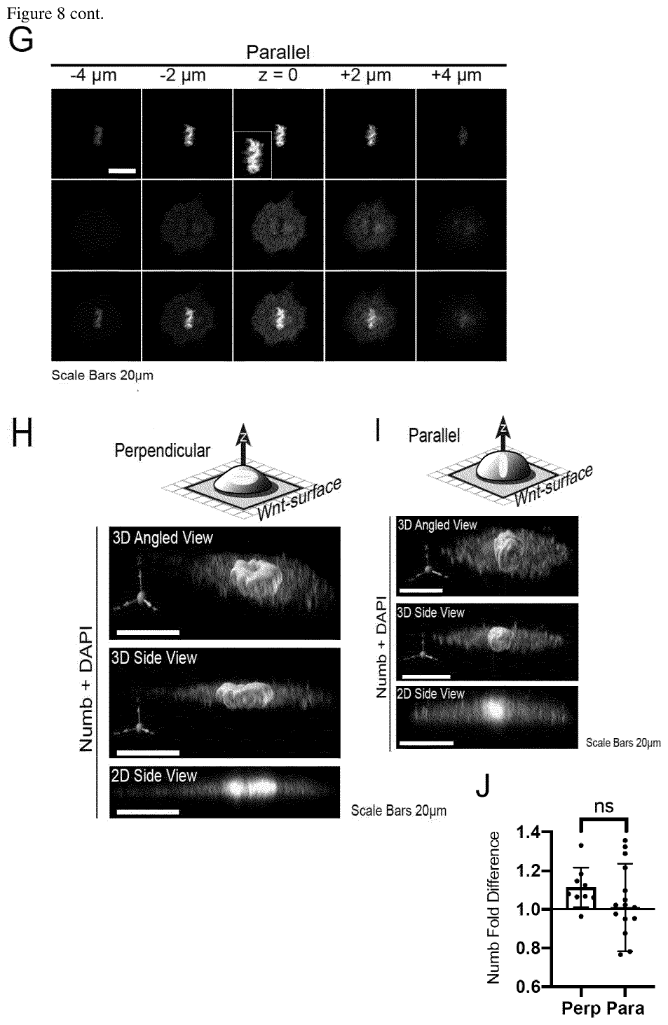

In a 2D system, localised Wnt3a can orient the spindle of murine embryonic stem cells (Habib, S. J. et al (2013) supra), so an investigation was carried out to see if localised Wnt3a can also orient the spindle of hSSCs in a 3D environment. On average 53% of hSSCs, isolated from the bone marrow, respond to Wnt3a and can activate the Wnt/β-catenin pathway (Lowndes, M. et al 2017 supra). hSSCs were cultured on a Wnt3a-platform and the cells overlaid with Collagen type 1 to create a 3D environment. The plane of mitotic division of hSSCs during the metaphase was visualised using 3D imaging and in silico reconstructions. Two categories of dividing cells could be identified: cells that divided perpendicular to the localised Wnt3a and align the chromosomes during metaphase in a flower-like chromatin ring when observed from above (axial resolution) (see FIG. 1D and FIG. 1E), or cells that divided parallel to the Wnt3a source and the chromatin ring, standing on edge, appears as an oblong shape when viewed from above (see FIG. 1H to FIG. 1I). The orientation of the plane of mitotic division was also confirmed by visualising the microtubules and the centrosomes (FIGS. 8A, B, D and E). Interestingly, a significant increase in cells that divide perpendicular to the Wnt3a platform was observed in comparison to inactivated Wnt3a-platform (iWnt3a) that was treated with DTT to break the disulphide bridges of Wnt3a, rendering the protein biologically inactive (see FIG. 1C) (Lowndes, M. et al (2017) supra; Lowndes, M. et al (2016) supra). The iWnt3a-platform was treated with DTT to break the disulphide bridges of Wnt3a, rendering the protein biologically inactive It was concluded that localised presentation of Wnt3a to one side of the hSSCs orients the axis of mitotic division in 3D, promoting divisions perpendicular to the Wnt3a source.

Specific spindle orientation often requires compartmentalisation of the polarity protein atypical Protein Kinase C ζ (aPKC ζ) to one side of the cell (Schlessinger, K. et al (2007) J. Cell Biol. 178: 355-361). Accordingly, in single perpendicularly dividing hSSCs, it was found that the aPKCζ was enriched in the Wnt3a-proximal half of the cell whereas, in parallel dividing cells, aPKCζ was distributed throughout the cell with minimal bias to either side (FIGS. 8C to 8E). Conversely, Numb, a Notch inhibitor implicated in ACD in some systems (Inaba, M. & Yamashita, Y. M. (2012) Cell Stem Cell 11, 461-469), was not polarised in the dividing hSSC, with comparable distribution to both sides regardless of division orientation (FIGS. 8F to 8J).

Single ESCs in contact with bead-tethered Wnt3a proteins polarised components of the Wnt3a/β-catenin pathway, such as β-catenin and APC towards the Wnt3a-bead (Habib, S. J. et al (2013) supra). One function of both proteins, among many in the cell, is the regulation of spindle orientation (Garcin, C. L. & Habib, S. J. (2017) supra). The distribution of both molecules in dividing hSSCs was visualised. In divisions perpendicular to the Wnt3a-platform, it was found that APC and β-catenin localise mainly to the Wnt3a-proximal half of the cell (see FIGS. 1D-to 1G and 1L). In divisions parallel to the Wnt3a source, both proteins were similarly distributed in both halves of the cell (see FIGS. 1H to 1L).

Taken altogether, localised Wnt3a proteins polarised aPKC (, APC and β-catenin in Wnt-responsive and dividing hSSCs and oriented the plane of mitotic division perpendicular to the Wnt3a source to promote ACD. This oriented division generated one Wnt3a-proximal hSSC, and a Wnt3a-distal cell primed to osteogenic differentiation that move upwards in the 3D collagen type 1 gel (FIG. 1B).

Immobilised Wnt3a Proteins Segregate Cell Fate Markers in Dividing hSSCs

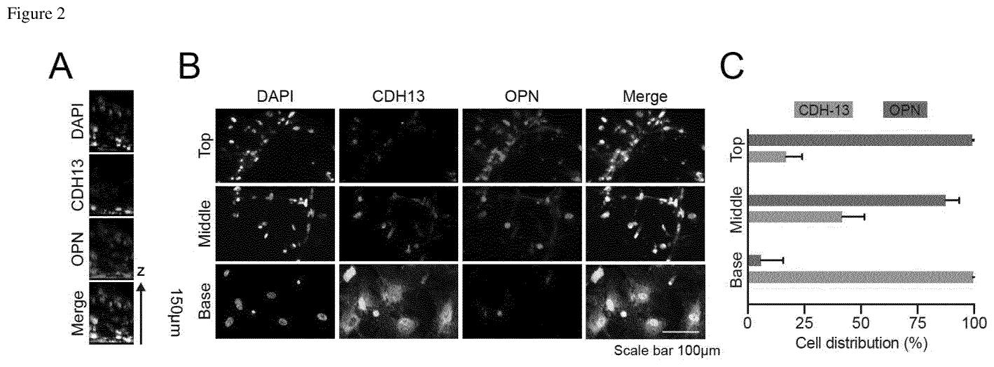

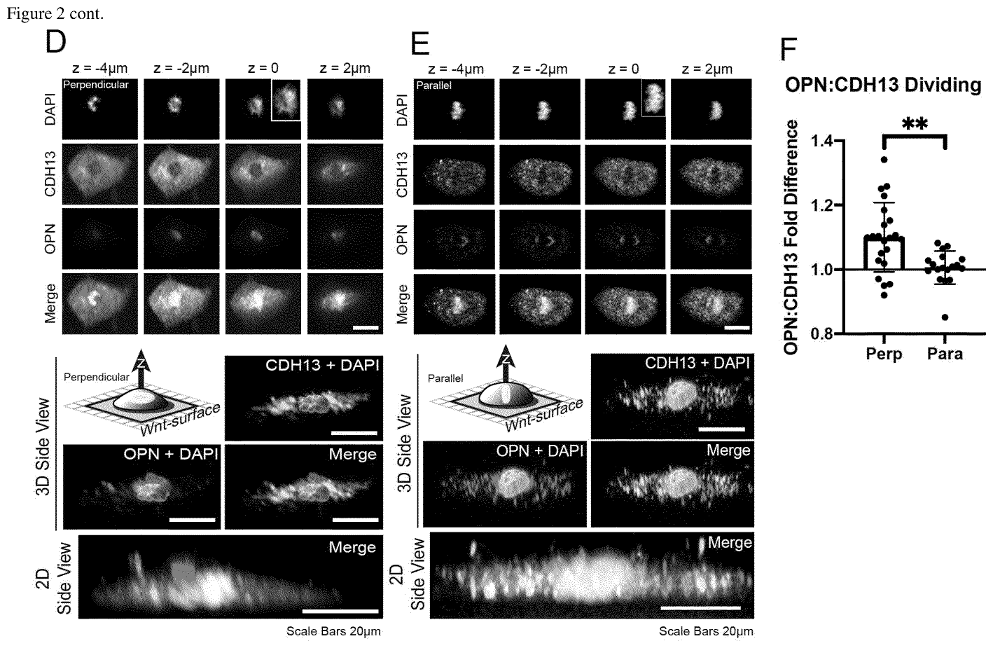

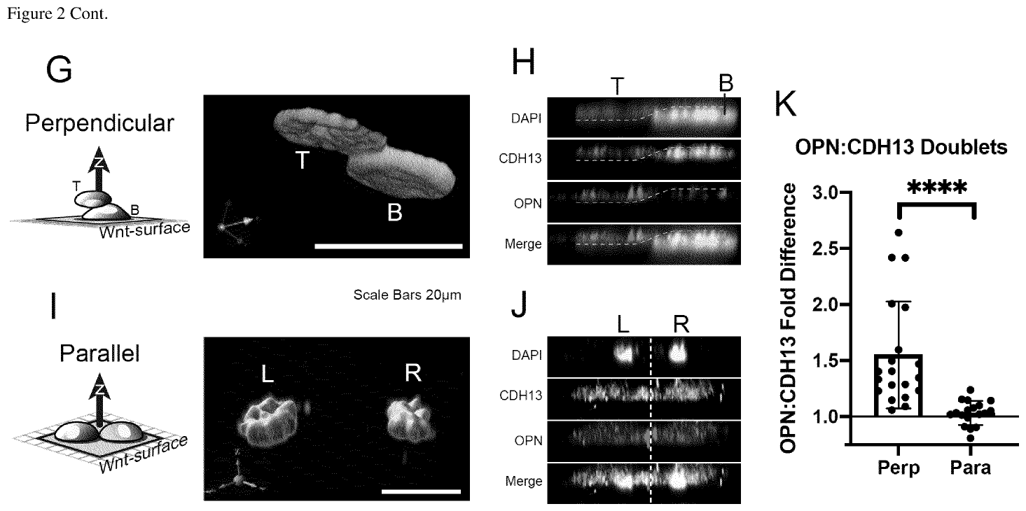

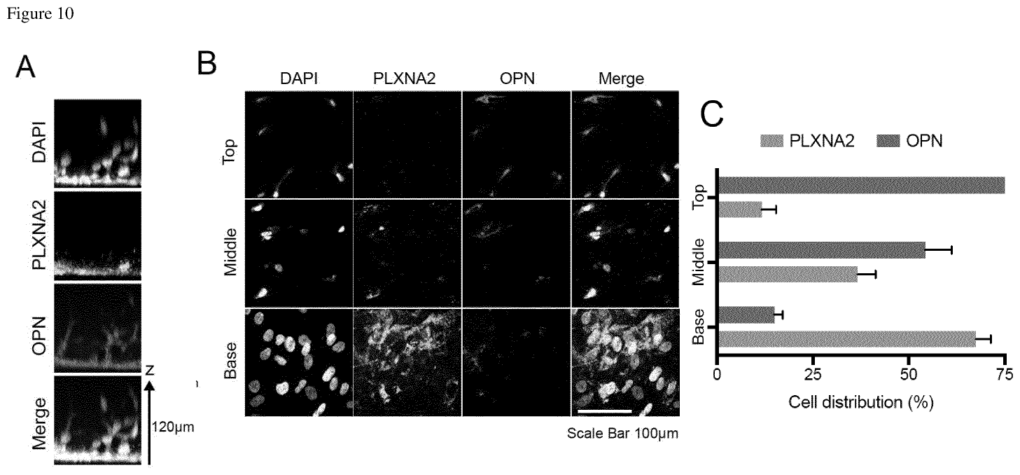

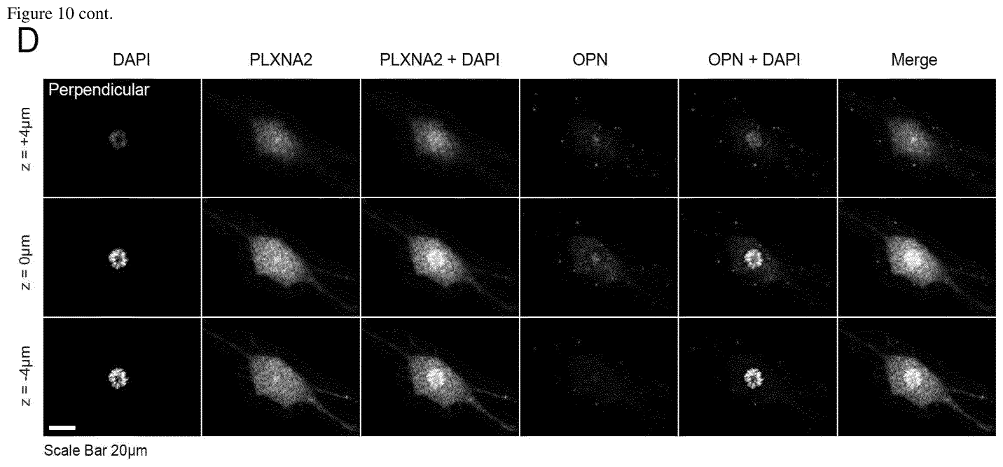

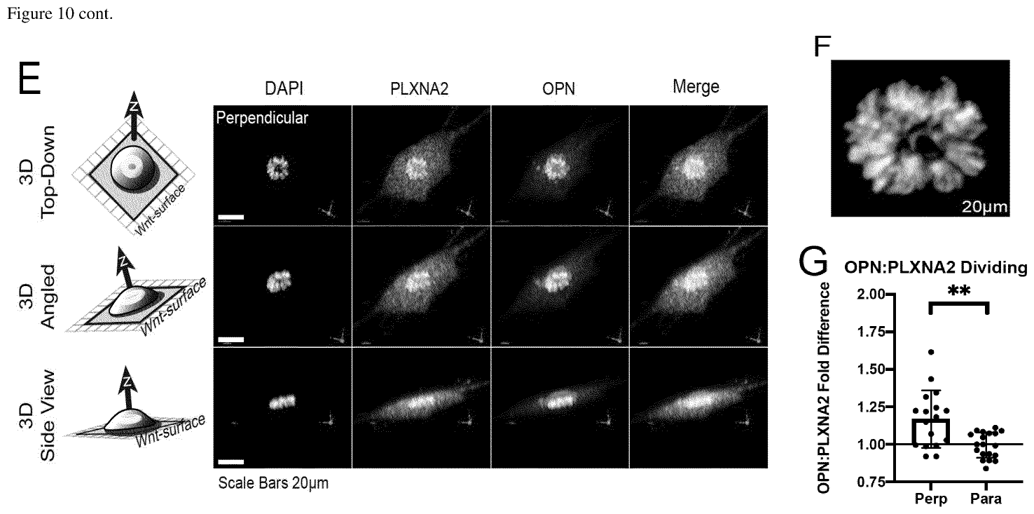

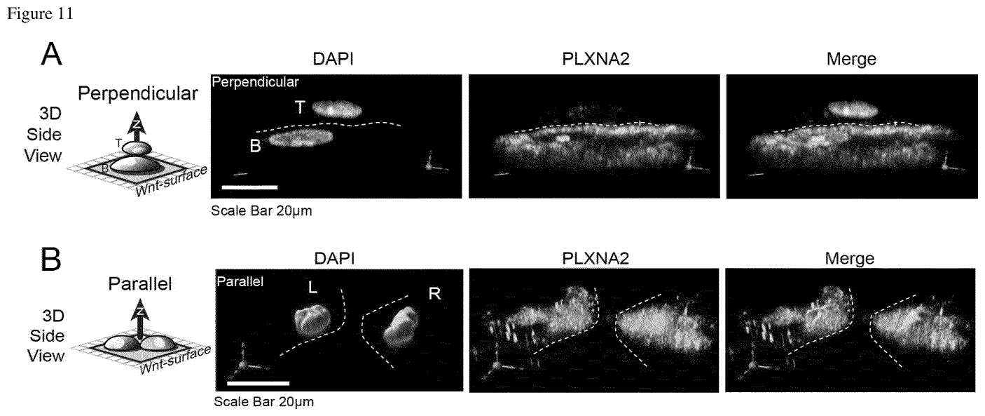

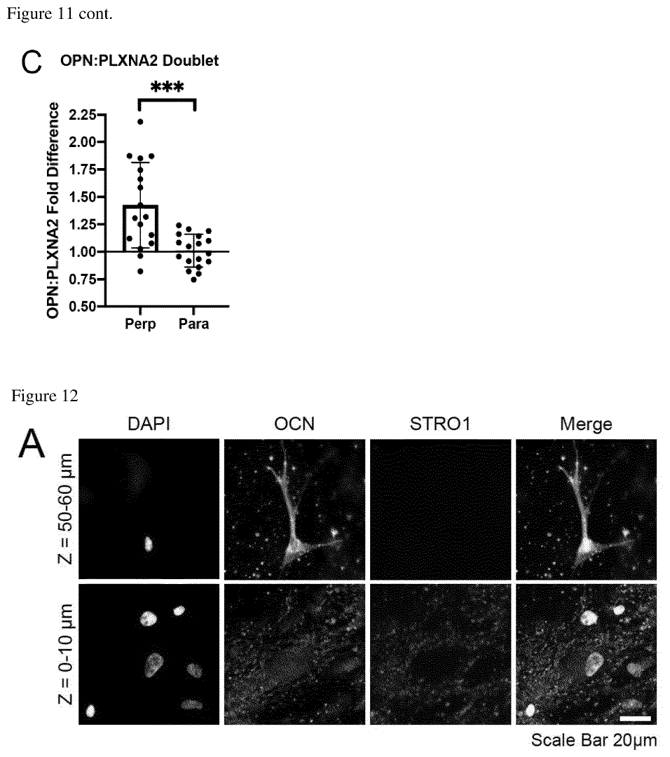

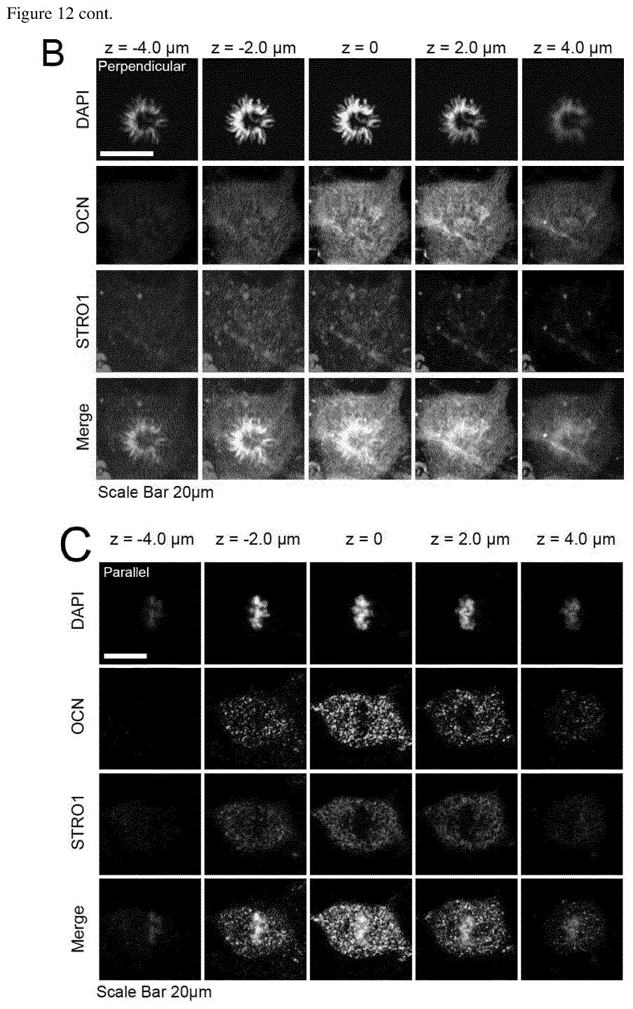

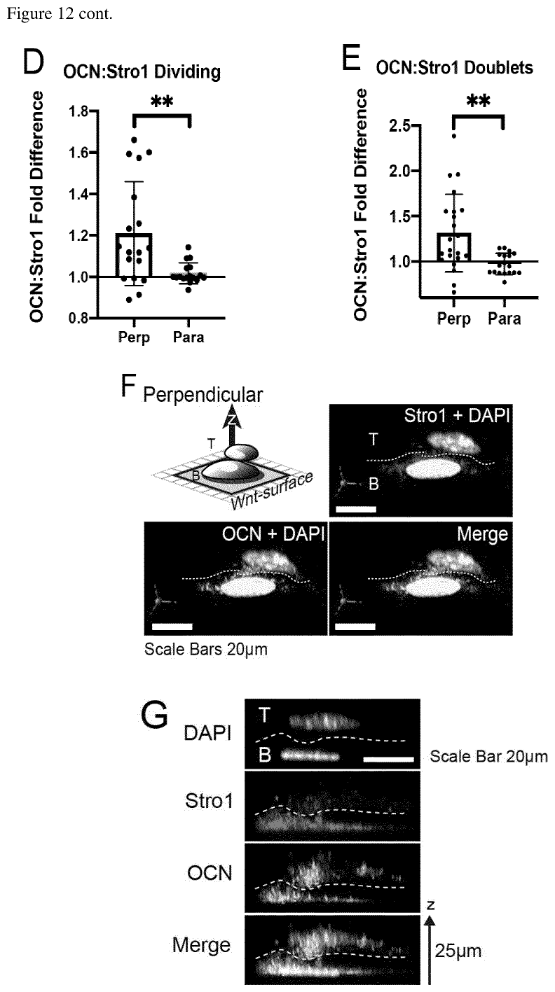

The Wnt3a-platform retained hSSCs that expresses high levels of the stem cell marker Stro1 and lower levels of the osteogenic cell fate marker Osteocalcin (OCN). Cells that migrated upwards in the 3D gel gradually downregulated Stro1 and upregulated OCN (FIG. 12A) (Lowndes, M. et al (2016) supra). Recently, proteomic and functional analysis of hSSCs isolated from the bone marrow demonstrated Cadherin-13 (CDH13) and the semaphorin co-receptor PLXNA2 as a novel hSSC marker (Holley, R. J. et al (2015) Stem Cell Reports 4: 473-488). Evaluation of the protein expression profile of CDH13, PLXNA2 and the early differentiation marker Osteopontin (OPN) (Zohar, R. et al(1998) Eur. J. Oral Sci. 106(1): 401-407) was carried out in the cells comprising the WIOTM. Similar to Stro1, CDH13 and PLXNA2 were highly expressed in Wnt3a-proximal cells and downregulated in cells that migrated farther from the immobilised Wnt3a. OPN showed similar expression pattern to OCN (FIGS. 2A to 2C and FIG. 12A). These markers further confirm the composition and osteogenic identity of the WIOTM. Since hSSCs co-express stem cell and differentiation markers, albeit at different levels, the distribution of these markers was monitored during cell division. Co-staining of CDH13 and OPN in Wnt3a-perpendicularly dividing hSSCs showed that the Wnt3a-proximal half had higher levels of CDH13 in comparison to the Wnt3a-distal half Conversely, OPN was enriched in the Wnt3a-distal half of the cell (FIGS. 2D and F and FIG. 9A). This segregation pattern of the markers was also observed in telophase and post-division (FIGS. 2G, 2H and 2K and FIG. 9B). In cells that oriented the mitotic plane parallel to the Wnt3a source, the stem cell and differentiation markers were similarly distributed between both halves of the cell (FIGS. 2E, 2F, 2I and 2K and FIG. 9C). The OPN:CDH13 ratio asymmetry of the dividing cells was confirmed by quantifying the relative expression of OPN:PLXNA2 and OCN:Stro1 in both mitotic single cells and post-mitotic cell doublets (FIG. 10D-10G, FIG. 11A-11C, and FIG. 12B-12G).

These results indicate that, in Wnt3a-oriented division of single hSSCs, localised Wnt3a also breaks the cellular symmetry. Localised Wnt3a induces the asymmetric distribution of components of the Wnt/β-catenin pathway and the polarity protein aPKC (that co-segregate with stem cell fate markers to the Wnt3a-proximal half of the cell. The Wnt3a-distal half of the cell has lower levels, if any, of aPKC ζ, β-catenin and APC, and is enriched in early osteogenic differentiation markers. Breaking the cellular symmetry in the metaphase prepares the cell for asymmetric stem cell division that produces two separate daughter cells: Wnt3a-proximal hSSCs with high levels of stem cell markers near the Wnt source and a Wnt3a-distal cell that expresses higher levels of osteogenic differentiation markers is directed away from the stem cell niche. In summary, the inventors' observation on the role of localised Wnt3a proteins to orient asymmetric cell division (ACD) from single murine embryonic stem cells in a 2D system (Habib, S. J. et al. (2013) Science 339, 1445-1448) to human adult stem cells in 3D culture has been extended. Importantly, it has been shown that localised Wnt is able to overcome differentiation cues from the media by maintaining the stem cell identity of Wnt-proximal cells. A crosstalk has been highlighted between Wnt machinery and the evolutionarily conserved intrinsic polarity cues to cooperate in regulating ACD.

Localised Wnt3a proteins drive ACD of hSSCs to generate the WIOTM, which recapitulate aspects of the human bone niche and might have therapeutic potential for bone repair. To test this, a biomimetic scaffold that delivers the WIOTM to a bone defect site was developed.

Fabricating Biodegradable Wnt3a-Bandage for Pro-Osteogenic Implant

The periosteum is a thin fibrocellular membrane (70-150 m) that surrounds bones throughout life (Squier, C. A. et al (1990) J. Anat. 171: 233-239). It has a diverse cellular composition including skeletal stem cells and osteoblast that contribute for the growth, remodelling and fracture repair of the bones. This experiment tested whether the WIOTM can recapitulate aspects of the periosteum in vivo in terms of long-term maintenance of the stem cell population and the ability to contribute to the formation of the mature bone cells.

Previous studies have used glass platforms for immobilising the Wnt3a proteins, which are not suitable for in vivo studies. To overcome this obstacle, a Wnt3a-bound bandage was engineered that can retain the transplanted cells, maintain their ‘stem-like’ properties and limit their distribution to the transplanted region. The scaffold requirements for bone tissue engineering include biocompatibility and degradability over an appropriate time scale (Alghazali, K. M. et al (2015) Drug Metab. Rev. 47: 431-454).

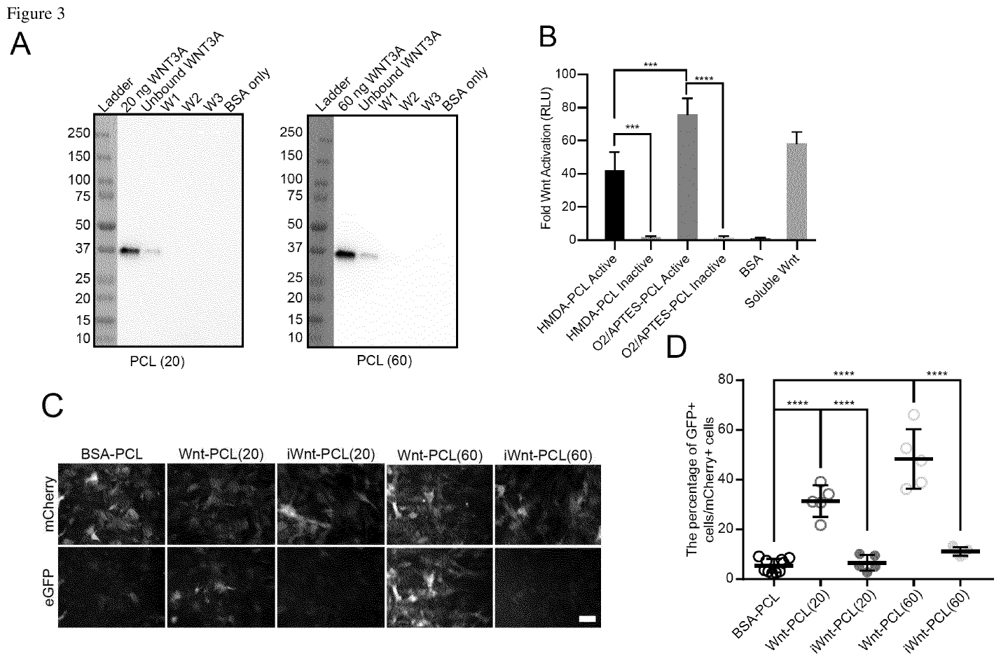

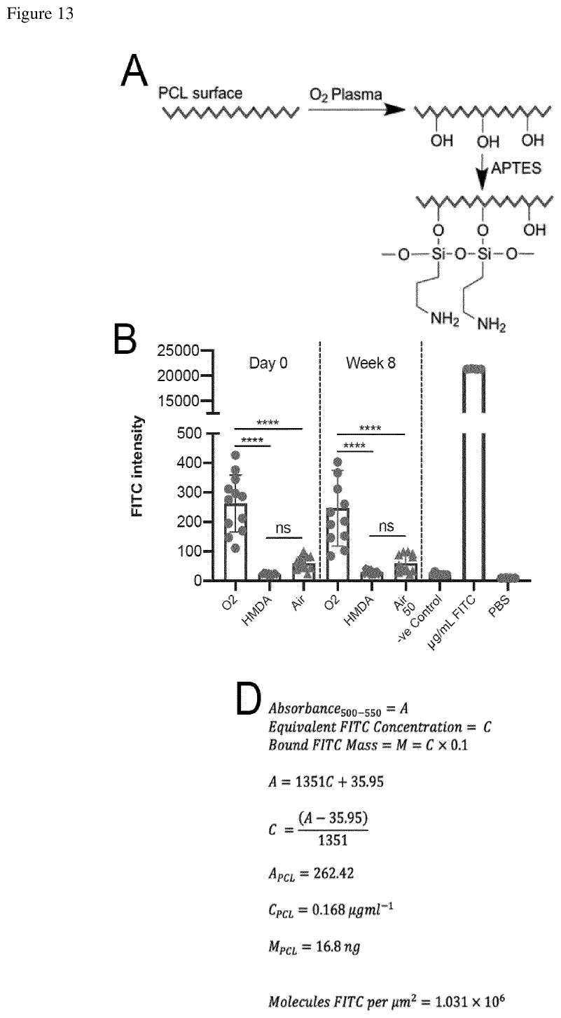

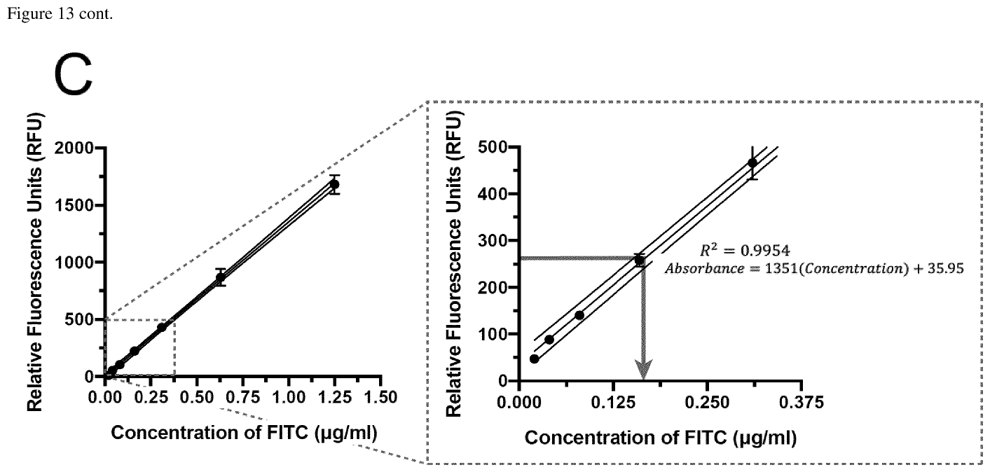

To generate the bandage scaffold, the clinically approved polycaprolactone (PCL) polymer was used to fabricate a film by solvent evaporation, and then the scaffold surface was oxygen plasma treated. This treatment provided primary amine functional groups for subsequent Wnt3a protein conjugation using aminopropyl-triethoxysilane (O₂/APTES) (FIG. 13A) (Wulf, K. et al (2011) J. Biomed. Mater. Re. Part B Appl. Biomater. 98: 89-100). It was found that the O₂/APTES surface functionalisation approach was more efficient than the conventional hexamethyldiamine (HMDA) approach (Zhu, Y. et al (2002) Biomacromolecules 3: 1312-1319). This was demonstrated by the approximately 10-fold higher Fluorescein isothiocyanate (FITC) intensity from the covalently bonding between FITC-isothiocyanate and primary amine functional group (FIG. 13B). Extrapolating from a standard curve of FITC intensity, it was estimated that 1.031×10⁶/m² of available primary amines can be crosslinked to Wnt3a ligands (FIGS. 13C and 13D). The primary amine functional groups on scaffolds remained stable over the cause of 8-week storage in Phosphate-buffered saline (PBS) at room temperature (FIG. 13B). Next, purified Wnt3a proteins were covalently conjugated using the previously described glutaraldehyde protein conjugation approach (Mills, K. M. et al (2017) supra; Lowndes, M. et al (2017) supra; Lowndes, M. et al (2016) supra). Wnt3a proteins bound efficiently to the surface as confirmed by αWnt3a Western Blot assay with minimal unbound Wnt3a proteins left in the system (FIG. 3A).

The functional activity of the immobilised Wnt3a proteins was then validated by assessing the Wnt/β-catenin signalling pathway activation. To do this, the TCF-luciferase reporter cell line (LS/L) assay was used and hSSCs transduced with 7×TCF-eGFP/SV40-mCherry reporter (Lowndes, M. et al (2017) supra; Lowndes, M. et al (2016) supra). The LS/L assay revealed significant increase in luciferase activity on the surface conjugated with active Wnt3a proteins, indicating the activation of Wnt/1 β-catenin signalling pathway (FIG. 3B). The luciferase activity was significantly decreased on inactivated-Wnt3a (iWnt3a) (Habib, S. J. et al (2013) supra). The O₂/APTES functionalisation method was more efficient in covalently binding Wnt3a proteins than the HMDA approach. This was reflected by the significantly higher fold activation of the Wnt/β-catenin pathway in LS/L cells cultured on O₂/APTES-Wnt3a-PCL films in comparison to the HMDA-Wnt3a-PCL films (FIG. 3B).

The biological activity of immobilised Wnt3a scaffolds was validated using hSSCs that harbour 7×TCF-eGFP/SV40-mCherry reporter. In this reporter, the production of the eGFP signal is under the control of 7TCF and is a signature of Wnt/β-catenin signalling pathway activation (Fuerer, C. & Nusse, R. (2010) supra). mCherry is constitutively expressed in the cells. Upon seeding these hSSCs onto the scaffolds, the cells exhibited dose-dependent eGFP expression increase (20 ng vs 60 ng; 31.3% vs 48.3%) (FIGS. 3C and 3D). The control films of covalently immobilised inactive Wnt3a proteins or the non-signalling molecule bovine serum albumin (BSA), showed significantly lower, if any, levels of eGFP.

As shown, the inventors have successfully developed a Wnt3a-bandage, made from a biocompatible material, which activates Wnt/β-catenin signalling pathway and can be delivered to in vivo, and generate the WIOTM for transplantation purposes.

Wnt3a- and WIOTM-Bandages Promote In Vivo Bone Repair

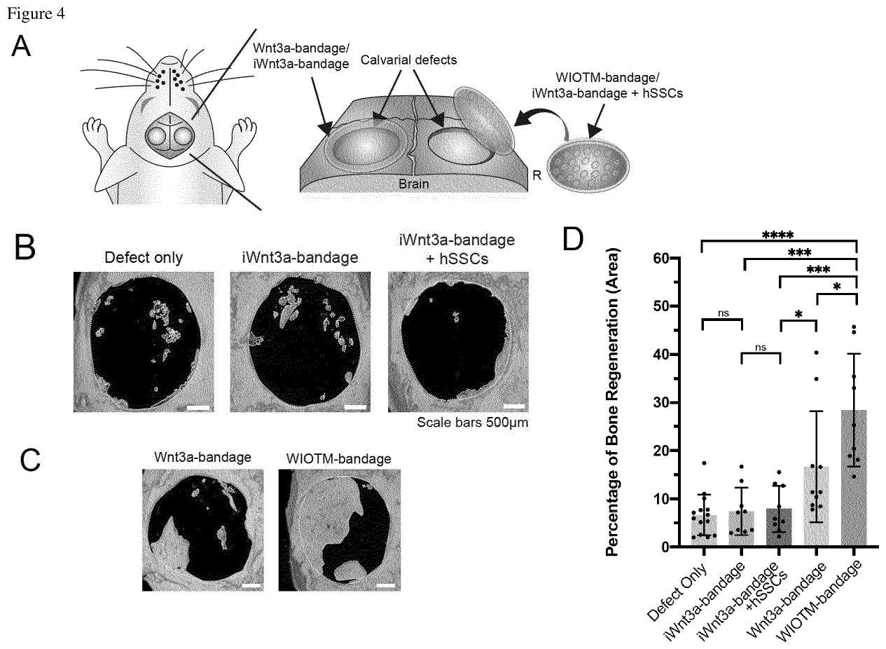

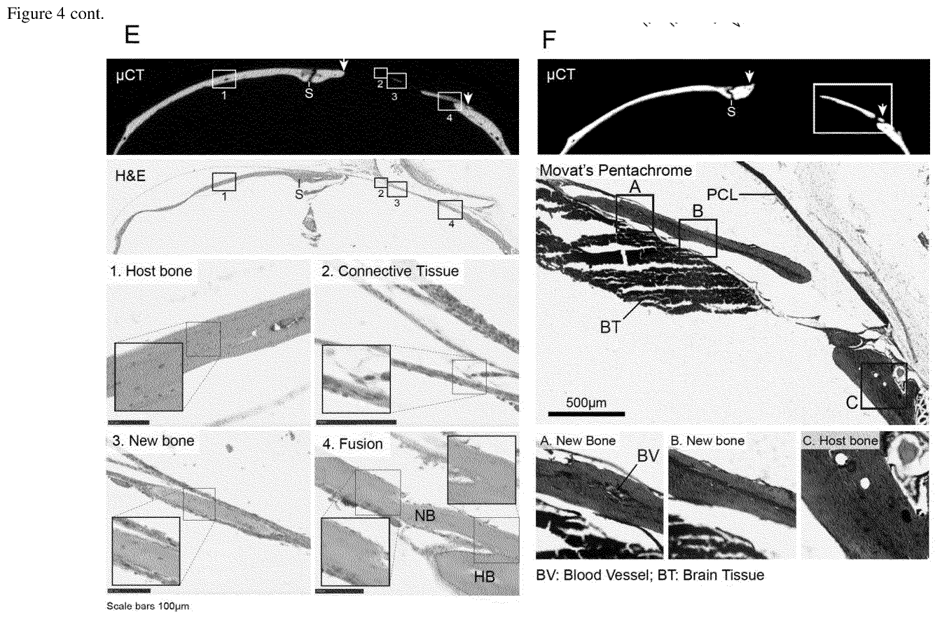

The critical-sized calvarial bone defect has been successfully used as a standardised and uniform model injury to evaluate bone repair by radiological and histological analysis (Gomes, P. S. & Fernandes, M. H. (2011) Lab. Anim. 45, 14-24; Samsonraj, R. M. et al (2010) PLoS ONE 5: e9370). The calvaria are flat bones that mainly form via intramembranous ossification. In this process, the overlying periosteum, the underlying dura mater and the cranial sutures provide osteoprogenitors for bone repair (Doro, D. H. et al (2017) Front Physiol. 8: 956). A subpopulation of these stem/progenitor cells is Wnt-responsive and contributes to bone formation and repair (Doro, D. H. et al (2017) supra; Zhao, H. et al (2015) Nat. CellBiol. 17: 386-396; Maruyama, T. et al (2016) Nat. Commun. 7: 10526; Wilk, K. et al (2017) Stem Cell Reports 8: 933-946). It was investigated whether the Wnt3a-bandage can stimulate endogenous bone repair on its own, and the ability of the WIOTM-bandage to integrate in vivo and contribute to the generation of new bone. Critical non-union defects were created with a 4 mm diameter in both or a single side of the parietal bone in thirteen-week old female severe combined immunodeficient (SCID) mice. A thin layer of collagen type1 was added into the defect, overlaid the Wnt3a- or the WIOTM-bandage on top of the defect sites and secured the bandages with medical glue (FIG. 4A). To determine the impact of the 3D osteogenic construct, the WIOTM-bandage was restricted to have less than 20,000 cells at implantation. Considering the number of cells found in the healthy calvarial bone of equivalent area (ca. 270k total cells per 12.6 mm²), this represents a stringent introduction of exogenous cells. This approach avoids outcomes and artefacts that may be attributed to high numbers of transplanted cells introduced to excess. Before surgery, all types of bandages were confirmed to be Mycoplasma-free. The bone repair was examined after 8-week time using micro-Computed Tomography (micro-CT) and histological analysis.

Micro-CT analysis of the defect sites without any implant, implanted with iWnt3a bandage, or implanted with iWnt3a-bandage cultured with hSSCs showed negligible healing after 8-week time periods (FIGS. 4B and 4D). These three controls had small superficial calcified tissues scattered over the defect area in an unorganised manner (covering an area of 6.6% for defect alone, 7.4% for iWNT3A-bandage and 7.9% for iWnt3a-PCL+ hSSCs). In contrast to the negative controls, defect sites overlaid by active Wnt3a-bandages demonstrated significant de novo formation of bone (covering a mean area of 17.67%) 8-weeks post-implantation. Furthermore, the WIOTM-bandage exhibited even more profound bone regeneration (covering an area of 28.4%; FIGS. 4C and 4D). A broad new bone area with high-density mineralisation was found to be converging from the defect bony edge to the core (FIG. 4C). Further evaluation using Haemotoxylin and Eosin (H&E) and Movat's Pentachrome stains showed that mineralised regions of newly forming bone on the defect site identified by micro-CT analysis demonstrated signatures of healthy bone, in which the nuclei appeared to be elongated, aligned, and sparsely distributed. Additionally, the surrounding calcified tissue presented with striations, representing the alignment of collagen fibres of the organic matrix and mineralisation characteristic of competent lamellar bone (FIGS. 4E and 4F). A connective/stromal-like tissue was also observed spanning the defect with densely packed nuclei (FIG. 4E). In active Wnt3a- and WIOTM-bandages treated defects, newly forming bone was present both in the mid-section of the defect site and in direct connection with the defect edge, which indicates bony fusion of new and existing bone in the process of repair and regeneration (FIG. 4E). Blood vessels within the newly forming bone were also detected (FIG. 4F).

The WIOTM-Bandage Maintains a Stem Cell Population and Contributes to Bone Formation in Calvarial Bone Defects.

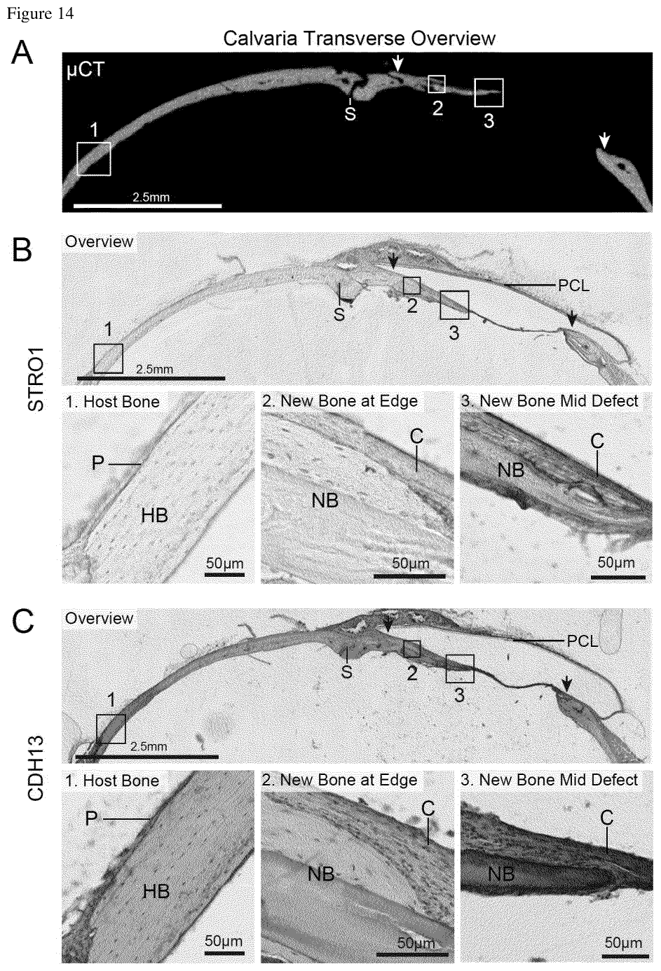

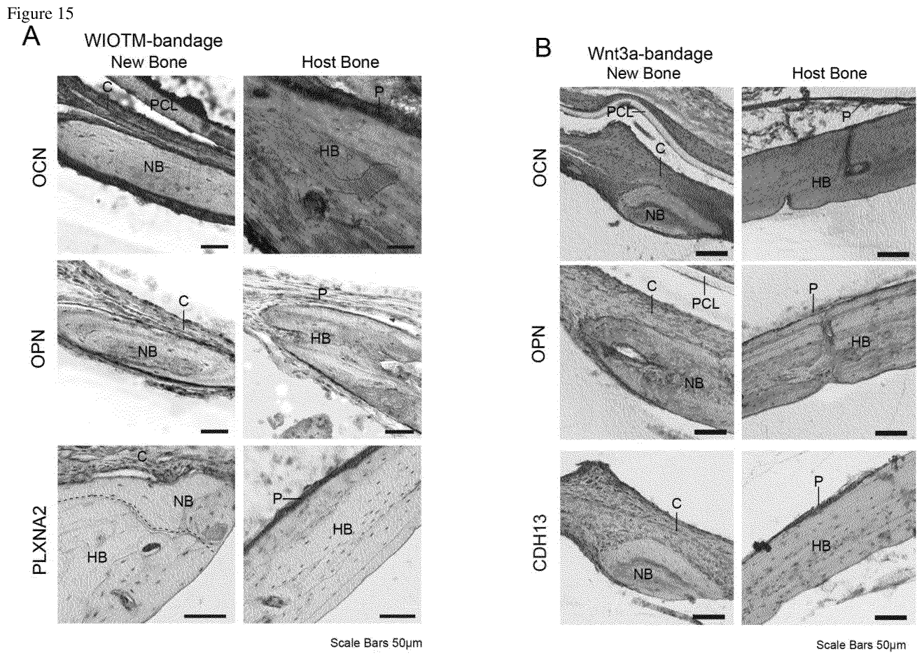

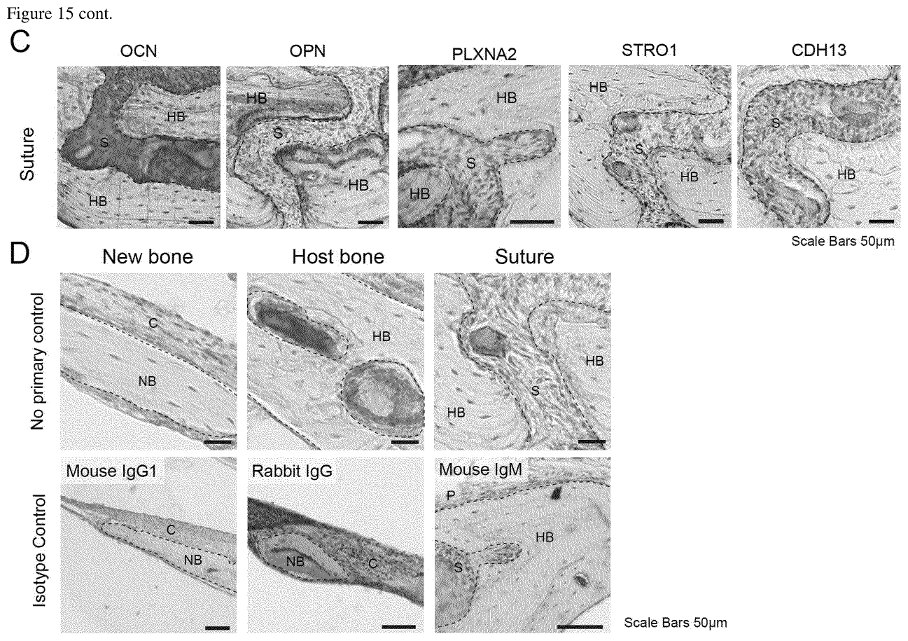

The integration of the WIOTM in the injury site and its contribution to the newly formed bone was investigated. Therefore, this experiment set out to characterise the anatomical distribution of cell identity markers that can distinguish between host and implanted cells. It was found that the hSSCs markers Stro1 and CDH13 and PLXNA2 were highly expressed in the WIOTM implanted area. Cells expressing these markers formed a connective/stromal-like tissue overlaying the newly forming bone as well as a thin layer surrounding it (FIGS. 14A to 14C and FIG. 15A). Stro1 staining in the host periosteum was minimal, potentially unspecific, and was mainly restricted to the margins of the outermost cell layer (FIG. 14B). Only background staining was seen in the sutures (FIG. 15C).

Faint CDH13 and PLXNA2 staining was observed in the host periosteum and the sutures (FIGS. 14A, 15A and 15C). Human and mouse CDH13 and PLXNA2 share 94.8% and 96.6% protein sequence identity respectively and, therefore, the antibodies seem to recognise proteins of both species. These results suggest that CDH13 and PLXNA2 are also expressed in the stem cell compartments of mouse calvaria.

The early differentiation marker OPN was expressed in the newly forming bone and the host bone and, to a lower extent, in the periosteum and the suture (FIGS. 15A and 15C). The higher deposition of OPN along bony edge of the osteoprogenitor compartment indicates that osteoblast and osteocytes were inducing matrix remodelling and bone expansion (Morinobu, M. et al (2003) J. Bone Miner. Res. 18: 1706-1715; Tsai, T.-L. & Li, W.-J. (2017) Stem Cell Reports 8, 387-400).

The osteogenic differentiation marker Osteocalcin (OCN) was abundantly expressed in the newly forming bone but was also expressed in the stem cell compartments (FIGS. 15A and 15C). These results are aligned with the previous observation where Osteocalcin is produced throughout the development from the late osteoblast to mature osteocytes (Bonewald, L. F. (2011) J. Bone Miner. Res. 26: 229-238). OCN is a secreted protein and therefore unlikely to localise solely in the calcified tissues.

Negative and Isotype controls confirm the specificity of the immunohistochemistry staining (FIG. 15D).

Immunohistochemistry provides qualitative insight into the structure and composition of the newly forming bone. However, the stem cell and differentiation markers used in these experiments can identify human and mouse cells. Therefore, human specific markers are required to quantify the WIOTM contribution to the newly forming bone. The Wnt/β-catenin signalling pathway is essential for the maintenance of hSSCs and WIOTM formation (Lowndes, M. et al (2016) supra), and it orchestrates bone repair in vivo (Doro, D. H. et al (2017) supra; Zhao, H. et al (2015) supra; Maruyama, T. et al (2016) supra; Wilk, K. et al (2017) supra). To better elucidate the impact of the WIOTM implant on bone regeneration, a WIOTM-bandage was generated using hSSCs that harbour 7×TCF-eGFP//SV40-mCherry reporter. The constitutively-expressed mCherry allows identification of human cells for linage tracing purposes. In some of the analysis, human 02-microglobulin (h3MG2) was utilised as a marker. Notably, staining h3MG2 showed an overlap with mCherry, indicating the specificity of the human markers (FIG. 16C).

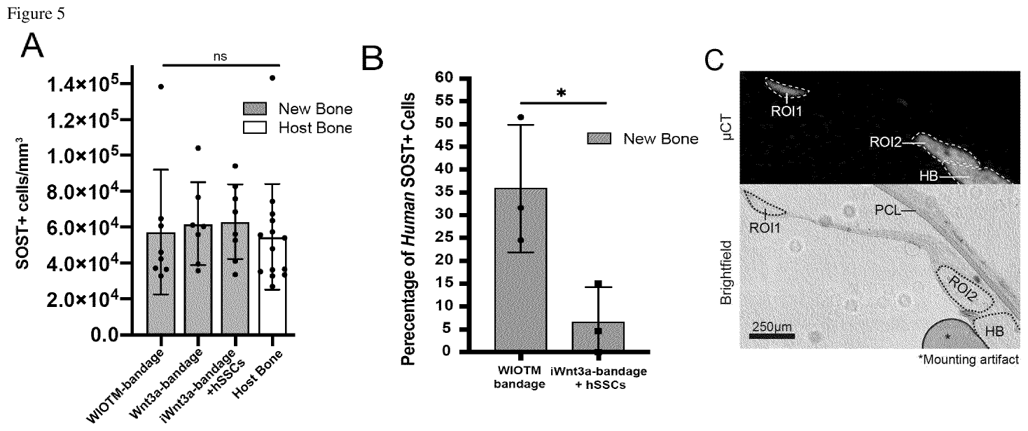

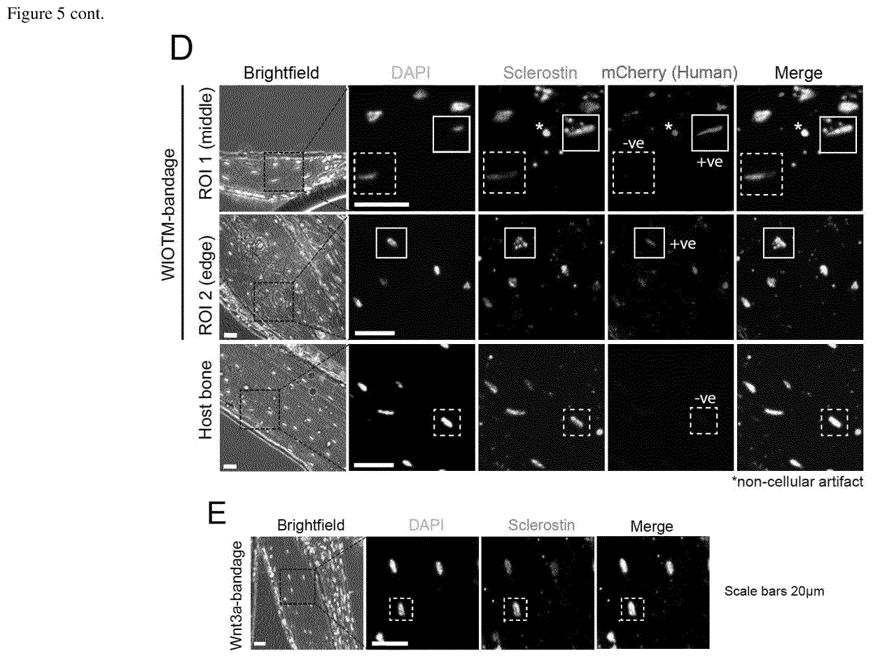

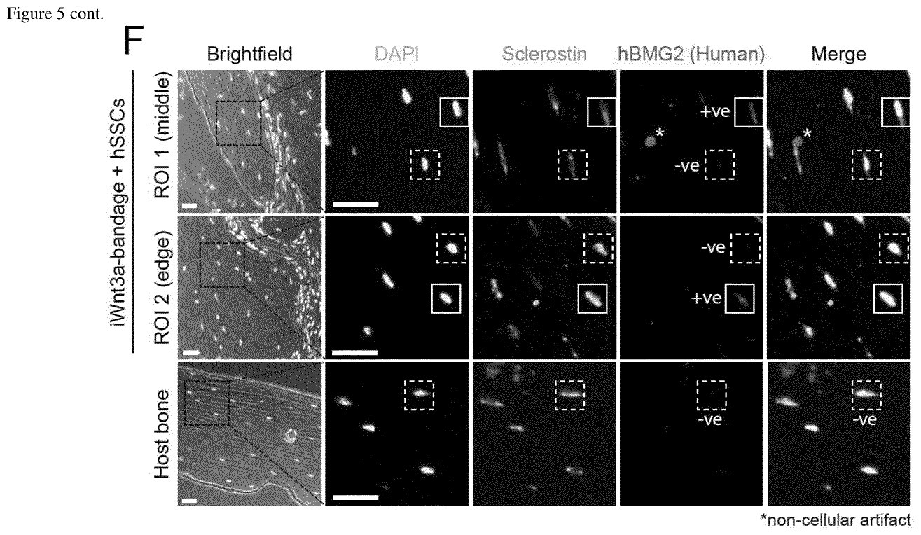

A method of thresholding immunofluorescence images of sample tissue based on control tissue signal intensity within the same sample was employed to allow identification of positive cells in tissue sections in situ (FIGS. 16A and 16B). This immunofluorescence analysis of areas of newly forming bone tissue showed that cells positive for sclerostin (SOST) were of equivalent density to healthy calvarial bone (FIGS. 5A and 5C to 5F, FIG. 16A). SOST is a marker expressed in mature osteocytes in advanced mineralization (Fan, J. et al. (2015) Tissue Eng. Part A 21, 2053-2065) and so represents the major cellular component of mature bone. In addition to mineralisation functions, sclerostin was also found to serve as the antagonist in the Wnt/β-catenin signalling pathway (Ramachandran, K. & Gouma, P-I. (2008) Recent Pat. Nanotechnol. 2, 1-7) that is critical in transitioning from osteoblast to osteocytes.

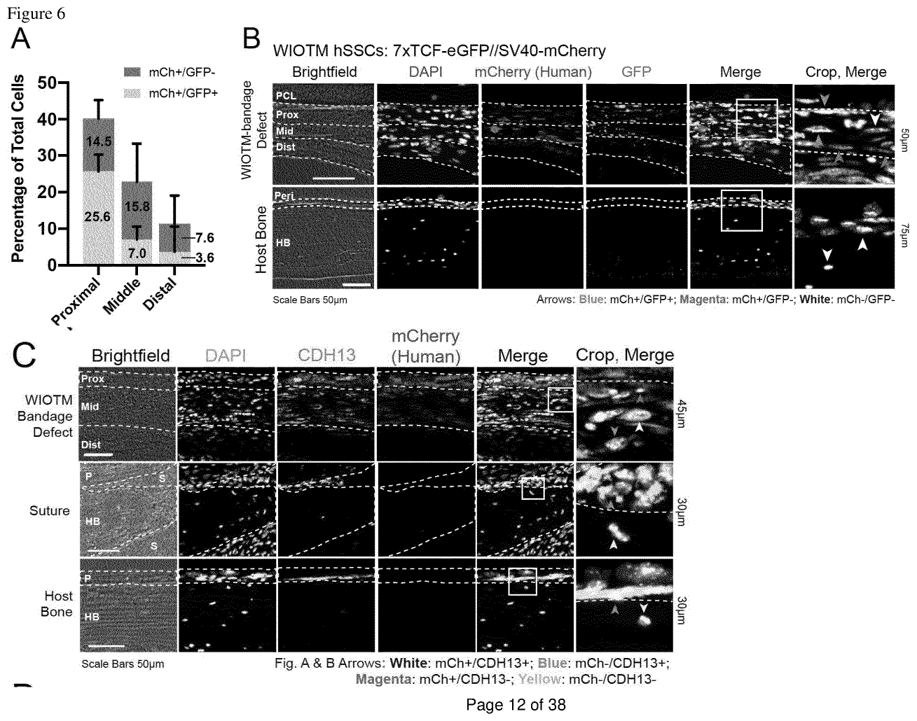

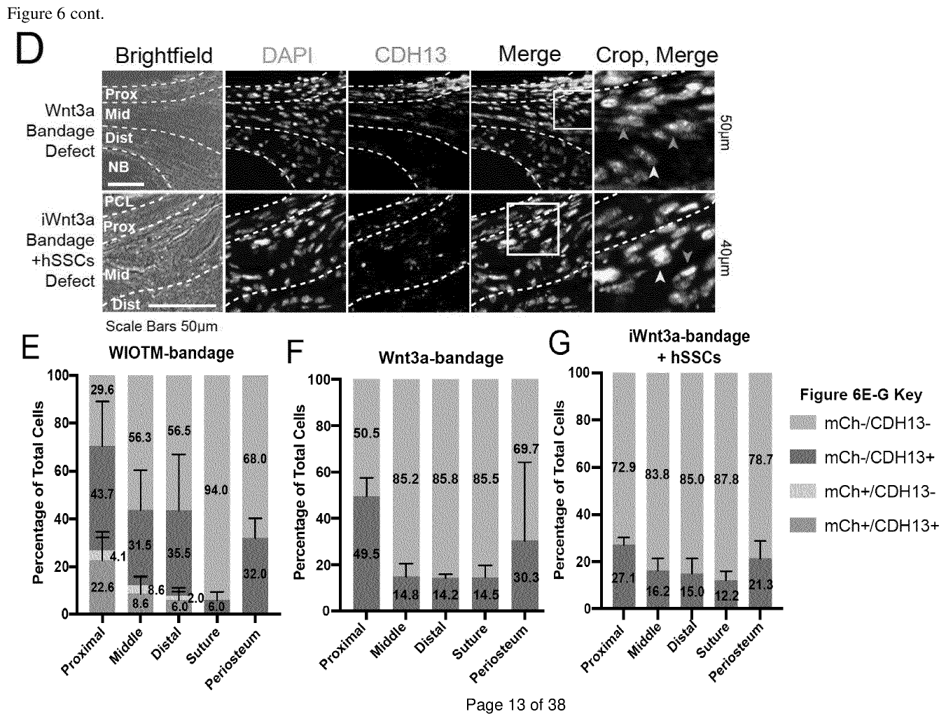

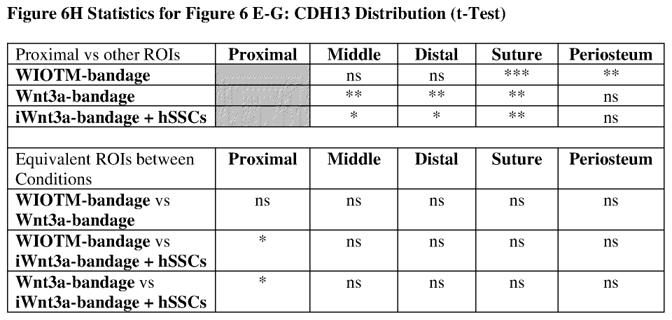

Lineage tracing revealed that, with WIOTM-bandage treatment, a significant proportion of sclerostin (SOST) positive cells were of human origin. In areas of new bone at the middle and edge of the defect, human mCherry positive cells represented 39.6% and 41.6% respectively of the total SOST+ population (ROI1 & ROI2, FIG. 5B), with an overall mean of 35.9% human-sourced SOST+ cells in new bone (FIGS. 5C and 5D). Implanted human cells were not detected in the neighbouring tissues of the host bone or sutures (FIG. 5D, FIG. 6C).

Conversely, hSSCs cultured on the inactive Wnt3a-bandage and overlaid with collagen gel did not integrate well into the minimal areas of new bone. In these samples human cells comprising only 6.5% of total SOST+ cells (FIGS. 5B, 5C and 5F), as identified by co-staining of the SOST+ cells with the human marker h3MG2. These results highlight the importance of the complete osteogenic structure over the implantation of stem cells alone in the survival, integration and contribution to bone repair in vivo. For this reason, it was crucial to perform an equivalent analysis on the connective tissue that supports the growth of the new bone.

The implanted hSSCs in the WIOTM expressed eGFP when the Wnt/β-catenin pathway was activated (see FIGS. 3C and 3D). The Wnt-responsive cells were predominantly in close contact with the immobilised Wnt3a-bandage: they represent 25.6% of total cells in the proximal region (63.8% of proximal human cells), but only 3.6% of total cells in the distal region (32.1% of distal human cells) (FIGS. 6A and 6B, FIG. 16B). Overall, 8-weeks post-implantation, 18.5% of the cells located throughout the connective/stromal-like tissue were of a human origin and expressed mCherry. On average 45.7% of these human cells were Wnt-responsive cells, (256 of 560 human cells, counted across sections from three animals), identified by eGFP expression.

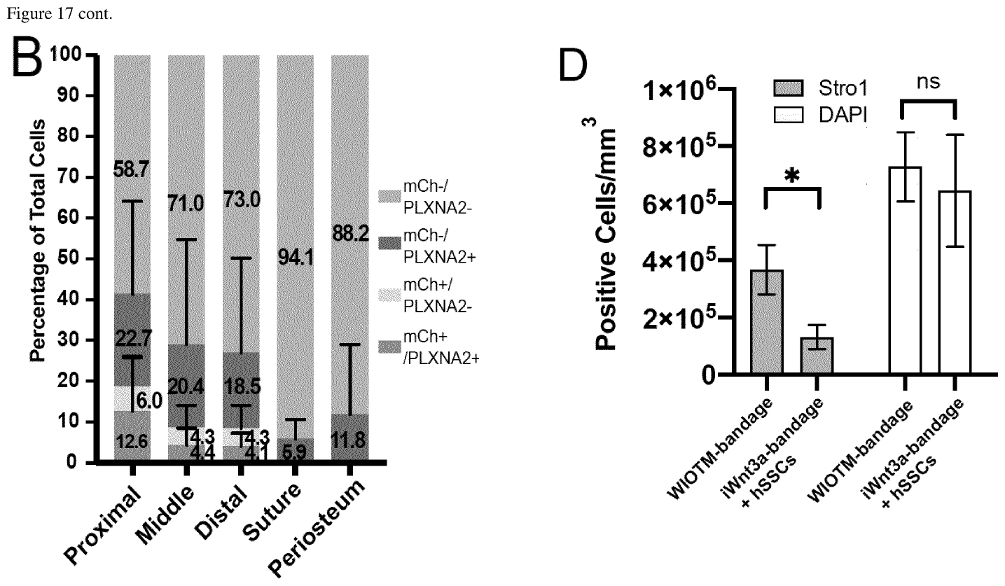

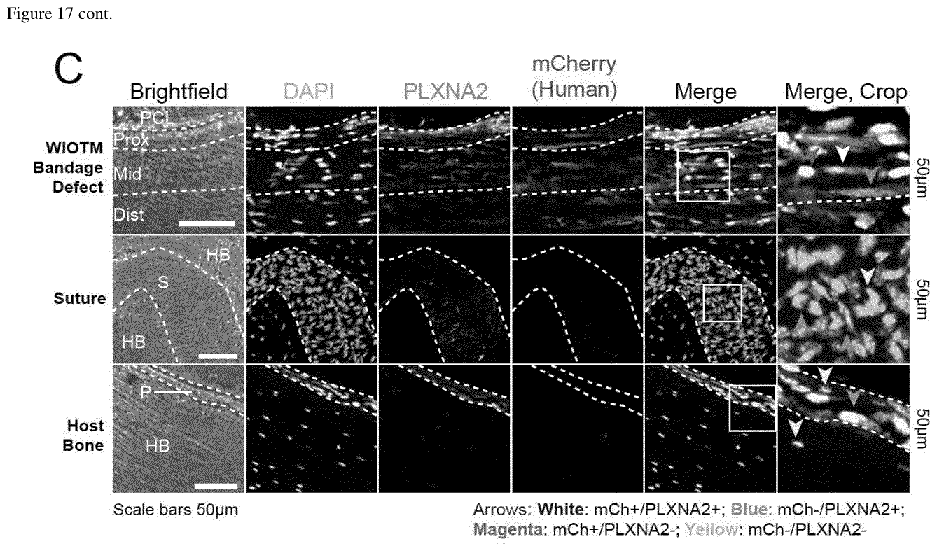

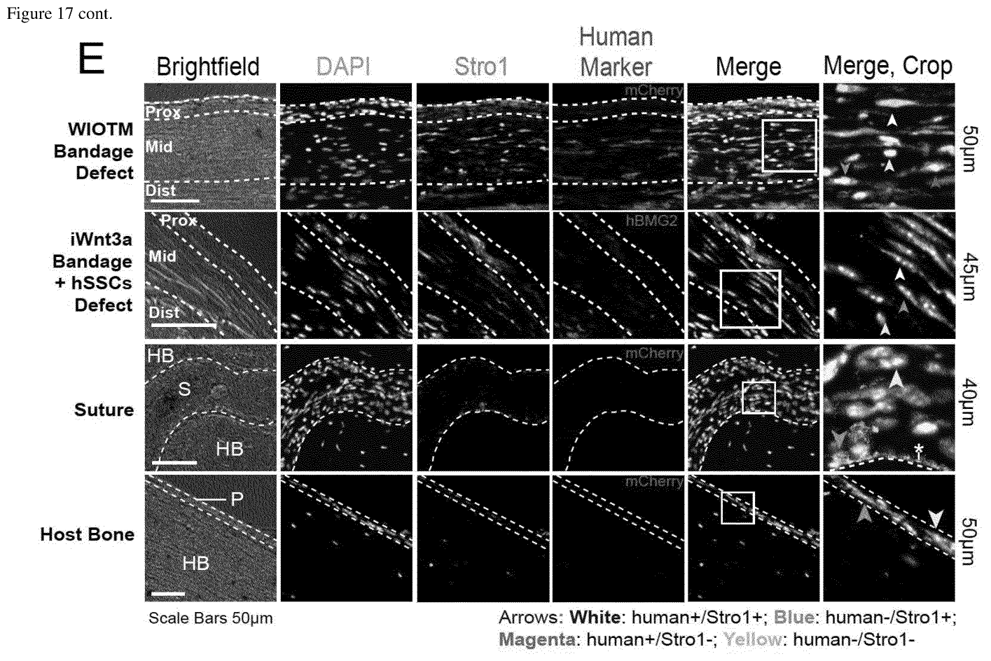

Next, the distribution of cells expressing stem cell markers in the connective/stromal-like tissue was investigated. The percentage of CDH13+ cells within the tissue was significantly higher proximal to immobilised active Wnt3a: 49.5% in Wnt3a-bandage, with only 27.1% in iWnt3a-bandage+ hSSCs (FIGS. 6D and 6F to 6H). This proportion of CDH13+ cells was further increased in the WIOTM-bandage, with a total of 66.3% CDH13 positive proximal cells. 22.6% of the cells were human CDH13+ cells (FIGS. 6C, 6E and 6H). Human CDH13+ cells were also detected in the WIOTM-bandage mid and distal regions, which also had further enrichment of total CDH13+ cells in comparison to Wnt3a-bandage or iWnt3a-bandage with hSSCs. A similar distribution of PLXNA2+ cells was seen in the WIOTM-bandage (FIGS. 17B and 17C).

In terms of cell density, this connective/stromal-like tissue (irrespective to the type of bandage treatment) was found to be similar to periosteum and suture mesenchyme, sources of endogenous SSCs/progenitors (DAPI, white bars; FIGS. 6C and 6D, FIG. 17A). However, the connective/stromal-like tissue in the Wnt3a- and WIOTM-bandages treatments had a significantly higher density of total CDH13+ cells (ca. 500,000/mm³ and 400,000/mm³ respectively) compared to host sources of CDH13+ cells in the periosteum or the suture (<˜200,000/mm³). This increased density of CDH13+ cells was not seen in the iWnt3a-bandage with implanted hSSCs (˜200,000/mm³), which was similar to the host periosteum (FIG. 6D, FIG. 17A). The significant differences between the increase in density of CDH13+ cells by active and inactive cellular bandages were seen also in the analysis of the hSSC marker Stro1 (FIGS. 17D and 17E). Considering all the results, our data suggest that Wnt3a-bandage increases the population of cells that express stem cell markers in the connective/stromal-like tissue. The WIOTM-bandage further contributes to this increase by supplying the tissue with human cells.

Immobilisation of Wnt on PLGA Bandage

The effectiveness of PLGA as a film for the WIOTM bandage has also been assessed. Methods are as set out above and are the same as those used for WIOTM bandages with PCL as a film.

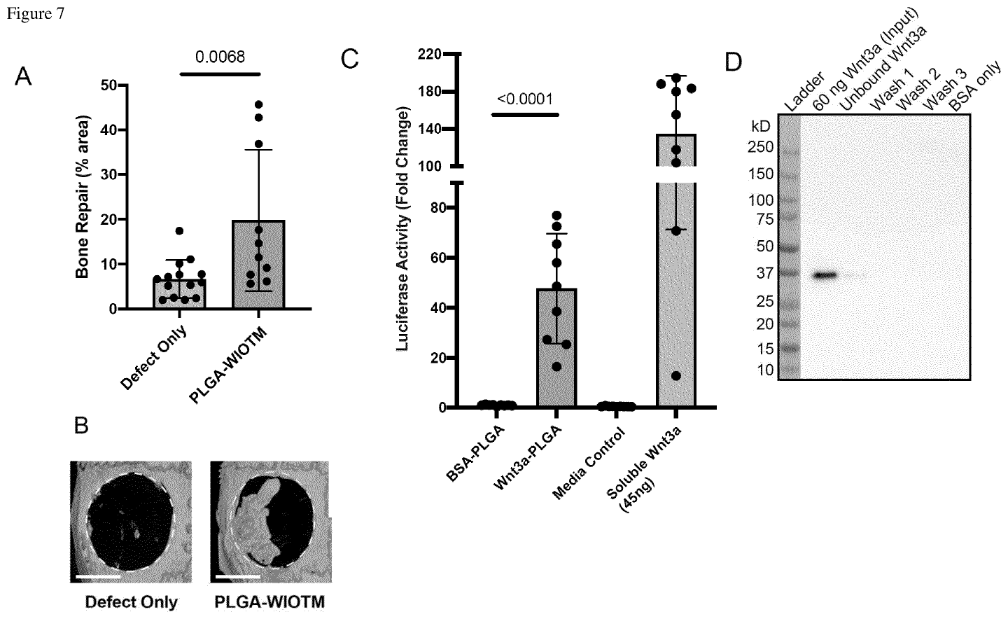

As shown in FIG. 7A, the percentage area of bone repair of a unilateral 4 mm diameter critical sized calvarial defect was quantified after 8 weeks, in 13 week old female SCID mice. Drilled defects were overlaid with collagen. In Defect Only animals (n=14), no further treatment was given. In the PLGA-WIOTM treated animals (n=10), poly(lactic-co-glycolic acid) (PLGA) Wnt3a-bandages were seeded with hSSCs and cultured for 7 days in a 3D osteogenic matrix in vitro prior to transplantation, to form the Wnt-induced human osteogenic tissue model (WIOTM).

FIG. 7B shows representative images of micro-Computed Tomography (microCT) scans of the calvarial defects, 8 weeks following surgery. Yellow dashed ellipse designates the original 4 mm diameter defect size.

FIG. 7C shows the fold change of luciferase activity as measured by Wnt/β-catenin reporter LSL cells harbouring the 7×TCF-luciferase construct, when seeded on PLGA bandages or tissue culture plastic. Readings from the conditions are: Wnt3a-PLGA (60 ng input Wnt3a immobilized on PLGA), Media Control (tissue culture plastic and standard culture media), and Soluble Wnt3a (tissue culture plastic and media supplemented with 45 ng soluble Wnt3a). All readings were normalised against BSA-PLGA (Bovine Serum Albumin immobilised on PLGA) and the results show data from, pooled from 3 independent experiments and n=9.

FIG. 7D is a representative Western blot showing 60 ng Wnt3a (equivalent to the amount input at the immobilisation step), Unbound Wnt3a (collected following immobilisation), Washes 1-3, and BSA (Bovine Serum Albumin) only.

The results demonstrate the successful immobilisation of Wnt on a PLGA bandage, as well as and the activation of Wnt/beta-catenin pathway in Wnt-reporter cell line. The WIOTM-PLGA bandage significantly improved the bone repair in a critical size calvarial defect after 8 weeks. One advantage of using PLGA, rather than PCL, is that PLGA has an in vivo biodegradability of about 28 days. In contrast, PCL biodegradability can last for years. As a result, PLGA is suitable for applications that require either short treatment or fast degradation of the bandage.

Taken all together, this is the first demonstration of simultaneous human SSC population maintenance alongside a cascade of differentiating osteogenic cells of a human origin in murine bone defect that promote bone repair. These findings also further signify the contrasting roles of the Wnt/β-catenin signalling pathway in orchestrating bone regeneration by recruiting and expanding Wnt3-responsive stem cells while also generating mature and functional bone cells.

The results show that the Wnt3a-bandage promoted enhanced endogenous bone repair in comparison to control bandages. Interestingly, the WIOTM-bandage further improved the bone repair by forming additional new bones that contained human mature osteocytes, while maintaining a Wnt-responsive stem cell population.

The results also show that localised Wnt3a induces asymmetric cell division (ACD) of hSSCs in 3D, which drives the WIOTM formation. In this process, during the metaphase, hSSCs polarise β-catenin and APC and the polarity protein atypical Protein Kinase C ζ (aPKC ζ) to the Wnt3a-proximal half of the cell and orient the plane of mitotic division perpendicularly to the Wnt3a source. Furthermore, it was found that stem cell marker CDH13 was enriched in the Wnt3a-proximal half of the cell, while the early differentiation protein marker Osteopontin (OPN) was enriched in the Wnt3a-distal half of the cell. The result of this division was a Wnt3a proximal cell with a hSSC cell fate and a Wnt3a-distal cell that is prone to osteogenic differentiation and is located within the 3D gel.

Discussion

Asymmetric cell division (ACD) is an evolutionarily conserved mechanism from bacteria to humans (Pereira, G. & Yamashita, Y. M. (2011) Trends Cell Biol. 21: 526-533). This process ensures the generation of diverse cell types, which is essential for the adaptation to the environment or development, patterning and maintaining tissues in multicellular organisms. ACD also regulates the number of stem cells in tissues. During mitosis, stem cells partition cell fate determinants and orient the mitotic spindle to maintain stem cell numbers while differentiated daughter cells are generated and proliferate.

Stem cell self-renewal is dependent on external signals that often include those of the Wnt family (Garcin, C. L. & Habib, S. J. (2017) supra). Wnt proteins are often secreted locally and presented to one side of the responsive cell (Mills, K. M. et al (2017) supra). In C. elegans, this localised action of Wnt proteins induces asymmetric cell division in embryonic and adult cells (Goldstein, B. et al (2006) Dev. Cell 10: 391-396). To mimic the in vivo situation, the inventors have previously immobilised Wnt covalently to synthetic surfaces and introduced them to mammalian stem cells (Lowndes, M. et al(2017) supra). Localised Wnt3a can induce oriented ACD of single mouse embryonic stem cells (mESCs) in 2D culture (Habib, S. J. et al (2013) supra). In this study, this observation has been extended and the inventors have demonstrated that localised Wnt3a could also induce oriented ACD of adult hSSCs in a 3D environment in media that supports differentiation. This localised and directional action of Wnt3a on hSSCs, but not soluble Wnt3a proteins that are globally added to the media, is essential for the WIOTM formation (Lowndes, M. et al (2017) supra). The results emphasise the evolutionarily conserved role of localised Wnt3 as in breaking cellular symmetry that can generate two daughter cells with different cell fate, a process that is essential for tissue formation and patterning (Mills, K. M. et al (2017) supra).

In the process of Wnt3a-induced ACD of hSSCs in a 3D scaffold, cells in the metaphase orient the axis of mitotic division towards the Wnt3a source. β-catenin and APC (components of the Wnt3a/β-catenin pathway), the cell polarity protein aPKCζ, and the stem cell markers CDH13 and PLAXN2 are enriched in the Wnt3a-proximal half of the dividing cell. In contrast, the Wnt3a-distal half of the cell has higher levels of the early differentiation markers OPN and OCN. This Wnt-mediated symmetry breaking of the dividing cell generates one Wnt3a-proximal hSSC cell and one Wnt3a-distal cell, localised in the gel, that initiates differentiation away from the Wnt3a source. Importantly, active Wnt/β-catenin signalling is required for the maintenance of the stem cell compartment in the WIOTM. The inventors have previously shown that blocking the pathway by IWR resulted in the loss of the expression of Stro1 and severely compromised the WIOTM formation (Lowndes, M. et al (2016) supra).

The Wnt-induced ACD of hSSCs provides unique opportunities to dissect molecular mechanisms and protein networks involved in early stages of human osteocytogenesis at high spatial/temporal resolution.

Initial observations identified localised Wnts as upstream regulators for CDH13 and aPKC(. Classical cadherins (including CDH13) and β-catenin can form complexes on the adherens junctions. These complexes can also regulate the availability of 3-catenin to participate in Wnt-mediated signalling. Loss of function mutations in classical cadherins leads to decreased cell adhesion, and an increase in cell motility (Nelson, W. J. & Nusse, R. (2004) Science 303: 1483-1487). Interestingly, the Wnt3a-proximal hSSC inherits high levels of β-catenin and CDH13, while the Wnt3a-distal cell has lower levels of β-catenin and CDH13 and moves upwards into the gel.

aPKCζ protein is a component of the evolutionary conserved machinery of the PAR complex. In many organisms and developmental stages, this machinery acts independently of external cues to regulate cell polarity and ACD (Rafiq, Q. A. et al (2013) Biotechnol. Lett. 35, 1233-1245). The findings presented herein that localised Wnt controls the location of aPKCζ is unique and provides the grounds to explore new cross-talks between both machineries to regulate cellular asymmetry.

Additionally, Protein Kinase C (PKC) signalling regulates osteogenic differentiation and bone formation (Lee, S. et al (2014) BMC Cell Biol. 15: 42; Jeong, H. M. et al (2012) Biochim. Biophys. Acta 1823: 1225-1232). Several isoforms of PKC proteins, including aPKCζ, interact with and stabilise the protein levels of the transcription factor Msx2 (Jeong, H. M. et al(2012) supra). Msx2 represses the transcriptional activity of the osteogenic transcription factors Runx2, Dlx3 and Dlx5 (Yoshizawa, T. et al (2004) Mol. Cell Biol. 24: 3460-3472; Zhang, H. et al (1996) Proc. Nat. Acad. Sci. 93: 1764-1769). Msx2 was also shown to reduce DKK1 expression and subsequently enhance Wnt signalling (Cheng, S.-L. et al (2008) J. Biol. Chem. 283: 20505-20522).

The role of Wnt/β-catenin signalling pathway in orchestrating bone regeneration is well documented in various models (Im, J.-Y. et al (2013) Lab Anim. Res 29, 196-203; Brennan, M. A. et al (2014) Stem Cell Res. ITher. 5, 114-15; Nakahara, H. et al (2009) Transplantation 88, 346-353; Garcin, C. L. & Habib, S. J. (2017)Results Probl. Cell Differ. 61, 323-350; Mills, K. M. et al (2017) Open Biology 7, 170140; Lowndes, M. et al ( ) Nat Protoc. 12, 1498; Lowndes, M. et al (2016) Stem Cell Reports 7, 126-137; Nimni, M. E. et al (1987) J. Biomed. Mater. Res. 21, 741-771; Habib, S. J. et al (2013) Science 339, 1445-1448; Schlessinger, K. et al (2007) J. Cell Biol. 178, 355-361). Aberration in Axin2 expression, which serves as the critical negative regulator for Wnt/β-catenin signalling pathway, was found to accelerate proliferation and cause earlier differentiation of progenitor cells in the mouse model (Mills, K. et al (2017) Open Biology 7, 170140; Habib, S. J. et al. (2013) Science 339, 1445-1448). Lineage tracing in the mouse model further delineates the Wnt-induced osteogenic regeneration following injury, where the Wnt-responsive stem cell population that resides in the suture mesenchyme is found to be key in driving expansion and differentiation (Im, J.-Y. et al(2013) supra; Brennan, M. A. et al (2014) supra). Pharmacological studies were thus developed to explore the therapeutic efficacy of Wnt proteins in promoting bone regeneration.

Wnt3a delivered by liposomal vesicles to injured tibia was identified to improve bone repair. Although μCT data is lacking, the authors show that Wnt3a-liposome treatment stimulates proliferation of skeletal progenitor cells, and thus accelerated osteoblast differentiation that are essential for bone growth (Schlessinger, K. et al (2007) supra). However, this protein-based approach relies on the non-targeted diffusion of the liposome in the injury site to reach Wnt-responsive cells that will promote and enhance bone growth. Other small molecule approaches which aim to modulate the Wnt/β-catenin signalling pathway in favour of osteogenesis by inhibiting glycogen-synthase-kinase-30 (GSK30) and peroxisome proliferator-activated receptor-7 (PPARγ) were found to be effective in osteoinduction (Lowndes, M. et al (2016) supra; Inaba, M. & Yamashita, Y. M. (2012) Cell Stem Cell 11, 461-469). These molecules are often delivered with porous osteoconductive scaffolds that physically bridge the defect but result in the formation of woven and porous bone tissues with poor mechanical properties. Although promising candidates, small molecules are often nonspecific and can activate or inhibit other signalling pathways (Lee, K. et al (2005) The Spine Journal; Holley, R. J. et al (2015) Stem Cell Reports 4, 473-488).

Even though pharmacological approaches using soluble drug delivery demonstrate a promising therapeutic effect by generating woven bone in the defect site, it also poses biosafety concern. For example, a soluble drug delivery approach may require multiple rounds of injections in the injured site to achieve the desired effect, increasing the risk of infection. The lack of specificity and localised action of these agents might trigger collateral damage such as activating/suppressing the response of undesired cell population(s). As a result, the architecture and function of the target tissue, and/or adjacent tissues that can receive the drug by diffusion, might be negatively affected. For example, over-activation of Wnt signalling can result in increased bone volume, abnormal bone density and pathological thickening of the bone (Zohar, R. et al (1998) Eur. J. Oral. Sci. 106 Suppl. 1, 401-407; Junaid, A. et al (2007) Am. J. Physiol., Cell Physiol. 292, C919-26; Squier, C. A. et al (1990) J. Anat. 171, 233-239; Hutmacher, D. W. & Sittinger, M. (2003) Tissue Eng. 9 Suppl. 1, S45-64).

Tumorigenesis is also a concern in patients that are prone to cancer. In many cancers, tumorigenic cells have mutations in downstream effectors of the Wnt/β-catenin pathway that activate the signalling cascade (Alghazali, K. M. et al (2015) Drug Metab. Rev. 47, 431-454). Administered agents that can reach these cells by diffusion and further activate the Wnt/0-catenin pathway might enhance the cancer progression. Therefore, a targeted delivery of the therapeutic agent that can lead to a controlled and localised activation of Wnt/β-catenin pathway to the target cells is essential for limiting side effects while promoting healing. The Wnt3a patch of the present invention achieves these criteria.

Wnt3a patches of the present invention deliver an osseous therapeutic effect by anchoring Wnt3a proteins onto the film using covalent bonding. This engineering design mitigates the risk of spontaneous protein leakage and the subsequent side effects of activating other signalling pathways, affecting undesired cell populations and inducing carcinogenesis, thus greatly increasing its potential for translational applications.

The in vivo degradation rate of PCL is slow and may require a number of years to be fully broken down (Yang, Y. & Haj, El, A. J. (2006) Expert Opinion on Biological Therapy 6, 485-498). In the present invention, because the PCL is overlaid onto the defect site and is not incorporated into the forming bone, it can be easily removed after the repair if required. Alternatively, other types of biocompatible materials that have a faster degradation rate may be used as a scaffold to immobilise Wnt3a and for in vivo delivery. For example, the in vivo degradation rate of Poly Lactic-co-Glycolic Acid (PLGA) can be as short as one month (Malikmammadov, E. et al (2018) J. Biomaterials Science, Polymer Edition 29, 863-893).

The ligands of several signalling pathways have been shown to promote bone repair, most potent being Bone morphogenic proteins (BMP) (Wulf, K. et al(2011) J. Biomed. Mater. Res. Part B Appl. Biomater. 98, 89-100). In comparison to the nano-scale amounts of Wnt proteins using the Wnt patch of the present invention, the currently known approaches administer supra-physiological doses of BMP (μg-mg) to promote effective bone repair. Unfortunately, these approaches have many undesirable side effects, including compartment syndrome and progressive myopathy and heterotrophic ossification, with some cases leading to life-threatening complications (Zhu, Y. et al (2002) Biomacromolecules 3, 1312-1319; Fuerer, C. & Nusse, R. (2010) PLoS ONE 5, e9370). Additionally, the reported study that investigated acellular approaches for bone repair was conducted on wild type adolescent mice (Gomes, P. S. & Fernandes, M. H. (2011) Lab. Anim. 45, 14-24). The skeleton of adolescent mice is not completely mature and has improved capability for bone healing in comparison to adult animals which were the focus of the present study (Samsonraj, R. M. et al (2017) Tissue Eng. Part C Methods 23, 686-693; Doro, D. H. et al(2017)Front Physiol. 8, 956). Furthermore, the present study used immunocompromised mice, which pose additional obstacles for healing. SCID mice lack the adaptive immune response, which regulates osteogenic differentiation and activity, including osteoblast maturation and the later mineralization (Zhao, H. et al (2015) Nat. Cell Biol. 17, 386-396). The results provided herein show that, even in the absence of these key cues, the Wnt3a patch of the present invention is still able to initiate significant endogenous bone repair. Additionally, unlike the unorganised woven bone produced by other approaches using porous osteoconductive scaffolds, the newly formed bone produced as a result of Wnt3a patch of the present invention is histologically comparable to healthy bone.

In many cases, endogenous repair is not sufficient to heal bone defects, such as in elderly patients or in those with medical comorbidities, including osteoporosis, diabetes and cancer, or in patients that require specific reconstructive surgeries after trauma (Wulf, K. et al (2011) supra). Cell-based therapy has the potential to tackle this limitation. However, a major challenge in this field is the maintenance of exogenous stem cells, including hSSCs, and their progeny in vivo. Several reports have shown that transplantation of even millions of exogenous hSSCs within advanced 3D osteoinductive scaffolds in a defect site did not improve their long-term survival. This was attributed to the hostile environment of the injury site (Maruyama, T. et al (2016) Nat. Commun. 7, 10526; Wilk, K. et al(2017) Stem Cell Reports 8, 933-946). The inventors hypothesised that an osteogenic construct that contains stem cells alongside osteogenic cells rather than stem cells alone would have improved capabilities to integrate in the injury site. It was reasoned that a 3D osteogenic construct should closely resemble aspects of the bone niche and improve cell survival and integration.

To investigate this, a Wnt-Induced human Osteogenic Tissue Model (WIOTM) was generated on engineered Wnt3a patches. To challenge the hypothesis and the exogenous human cells, the patch was engineered to contain fewer than 20,000 cells, a comparatively low cell number compared to the area of the bone defect and to other reported cell-therapies using the same injury model (Krause, U. et al (2010) Proc. Natl. Acad Sci. U.S.A. 107, 4147-4152; Zeitouni, S. et al (2012) Sci. Transl. Med 4, 132ra55-132ra55; Hyun, J. et al (2013) Stem Cells Transl. Med 2, 690-702; Tsai, T.-L. & Li, W.-J. (2017) Stem Cell Reports 8, 387-400; Levi, B. et al (2012) Proc. Natl. Acad Sci. U.S.A. 109, 20379-20384; Brennan, M. A. et al (2014) Stem Cell Res. Ther. 5, 114-15; Nakahara, H. et al (2009) Transplantation L8, 346-353; Levi, B. et al (2010) PLoS ONE 5, e11177; Lough, D. et al (2017) Plast. Reconstr. Surg. 139, 893-905; Fan, J. et al (2015) Tissue Eng. Part A 21, 2053-2065). To reduce the regenerative contribution from the passive host cell migration, driven by osteoconductive materials, PCL film was generated using a moulding method that produces a relatively smooth surface compared to electrospinning that yields a nanofibrous surface that favours osteoblast migration (Ramachandran, K. & Gouma, P.-I. (2008) Recent Pat. Nanotechnol. 2, 1-7; Gao, Y. et al (2017) Sci. Rep. 7, 947). The form factor and mechanical properties of the implant were designed to be conducive to precise and minimally invasive in cranial surgery. The conventional bone graft mineral, hydroxyapatite, which is often used to bridge a defect and enhance woven and porous bone formation, was also eliminated (Im, J.-Y. et al (2013) Lab. Anim. Res. 29, 196-203; Chung, M. T. et al (2013) Tissue Eng. Part A 19, 989-997; Quinlan, E. et al (2015) J. Control Release 207, 112-119). Therefore, the fabrication design illuminates the impact of the inventive construct on bone repair.

Upon implantation into critical size calvarial defects in immunocompromised mice, both Wnt3a- and WITOM-patches significantly improved bone regeneration. Importantly, the patch design revealed another advantage in that the newly forming bone was histologically closer to healthy bone in comparison to the often reported woven and gapped new bones induced by porous scaffolds. The WIOTM cellular implant maintained the stem cell markers Stro1, CDH13 and PLAXN2 and close to the patch even after eight weeks in vivo and produced mature bone cells that contributed to the generation of mineralised bone. Detailed analysis shows that human cells constitute on average 18.5% of cells in the connective/stromal-like tissue inside the defect and 35.9% of SOST positive mature bone cells in the newly forming bone. Importantly, 64% of the cells positive for human stem cell markers in proximity to the Wnt3a surface had Wnt/β-catenin pathway activity, as made evident by GFP-positive staining. It is important to note that not all GFP-positive cells indicate active Wnt/β-catenin pathway at sample fixation, as the half-life of GFP in mammalian cells is 26 hours (Corish, P. & Tyler-Smith, C. (1999) Protein Eng. 12, 1035-1040). Therefore, it is important to note that not all GFP-positive cells indicate active Wnt/β-catenin pathway before the sample fixation.

The present study provides the grounds for studying human osteocytogenesis at the single cell level using high spatiotemporal resolution and allows for the identification of new molecular markers and pathways that are essential for this process. By using high throughput imaging, the invention can also provide a platform for drug screening to modulate human bone formation and for toxicity studies. Finally, by covalently immobilising Wnt3a on a biocompatible and biodegradable film, an acellular Wnt3a bandage/patch was generated that promoted endogenous healing of critical size bone defects. The osteogenic tissue model of the invention can also be generated on a 3D cellular scaffold, facilitating the maintenance of human osteogenic cells in vivo and be used to accelerate the bone repair.

1. A biological tissue-regeneration patch comprising:
i) one or more proteins from the Wnt family, or an agonist of the Wnt signalling pathway; and
ii) a scaffold,
wherein the one or more Wnt proteins or Wnt agonist is immobilised on the scaffold, and
wherein the scaffold is formed from a functionalised biocompatible polymer.

2. The patch of claim 1, wherein the one or more Wnt proteins is Wnt3.

3. The patch of claim 1, wherein the one or more Wnt protein or Wnt agonist is immobilised on the scaffold via covalent conjugation.

4. The patch of claim 1, wherein the one or more Wnt protein or Wnt agonist is immobilised on the scaffold via primary amine functional groups.

5. The patch of claim 1, wherein the biocompatible polymer is biodegradable.

6. The patch of claim 1, wherein the biocompatible polymer is formed as a film.

7. The patch of claim 1, wherein the biocompatible polymer is poly (ε-caprolactone) (PCL), poly (lactic-co-glycolic acid) (PLGA), poly (lactide acid) (PLA), Poloxamer, Povidone, Polydioxanone (PDS), Poly(vinyl alcohol) (PVA), poly(glycolide-co-caprolactone) (PGCL), Poly(l-lactide-co-ε-caprolactone) PLCL, poly-L/D-lactic acid (PLDLA), or alginate.

8. The patch of claim 1, wherein the biocompatible polymer is functionalised with primary amine groups.

9. The patch of claim 1, wherein the patch further comprises stem cells cultured on the biocompatible polymer.

10. The patch of claim 9, wherein the stem cells are cultured as a monolayer.

11. The patch of claim 9, wherein the stem cells are overlaid with an extracellular protein.

12. The patch of claim 11, wherein the extracellular protein is collagen.

13. The patch of claim 9, wherein the stem cells are human skeletal stem cells (hSSCs).

14.-18. (canceled)

19. A method of treating a defect, hole, niche, tear, or fracture in biological soft tissue, connective tissue, or bone in a subject, the method comprising applying a patch of claim 1 to said defect, hole, niche, tear, or fracture.

20. A method of manufacturing a biological tissue-regeneration patch as claimed in claim 1, the method comprising:
i) preparing a film from a biocompatible polymer;
ii) functionalising the surface of the polymer; and
iii) conjugating one or more proteins from the Wnt family, or an agonist of the Wnt signalling pathway, to the polymer surface.

21. The method of claim 20, wherein the surface of the polymer is functionalised to provide primary amine functional groups

22. The method of claim 20, wherein the surface of the polymer is functionalised by treatment with oxygen plasma and aminopropyl-triethoxysilane (APTES).

23. The method of claim 20, further comprising culturing stem cells after conjugation of Wnt, or an agonist thereof, onto the functionalised polymer surface.

24. The method of claim 23, wherein the stem cells are cultured as a monolayer.

25. The method of claim 23, wherein the stem cells are cultured for 5-10 days to generate a 3D structure.

26. The method of claim 23, wherein the method further comprises overlaying a layer of collagen over the stem cells.

27. A biological tissue regeneration method, comprising implanting the patch of claim 1 across or over a defect, hole, niche, tear, or fracture.

28. The biological tissue regeneration method according to claim 26, wherein the defect, hole, niche, tear, or fracture is a bone fracture.

29. The patch of claim 1, wherein the one or more Wnt proteins is Wnt3a.
